(19) United States
(12) Patent Application Publication
(10) Pub. No.: US 2022/0091102 A1
(43) Pub. Date: Mar. 24, 2022

(54) Screening for Inhibitors of Prostaglandin E Synthase 3 Useful for Treatment of Prostate Cancer

(71) Applicant: The Regents of the University of California, Oakland, CA (US)

(72) Inventors: Luke Gilbert, San Francisco, CA (US); Felix Feng, San Francisco, CA (US); Haolong Li, San Francisco, CA (US)

(21) Appl. No.: 17/544,527

(22) Filed: Dec. 7, 2021

Related U.S. Application Data
(63) Continuation of application No. PCT/US2021/015459, filed on Jan. 28, 2021.
(60) Provisional application No. 62/967,706, filed on Jan. 30, 2020.

(51) Int. Cl. G01N 33/50; C07K 14/46; C07K 14/72

(52) U.S. Cl. CPC G01N 33/5041; C07K 14/46; C07K 14/721; G01N 2333/99; C07K 2319/60; G01N 2333/723; G01N 33/5023

(57) ABSTRACT

Methods and compositions are provided for screening candidate agents for inhibition of prostaglandin E synthase 3 (PTGES3) and anti-cancer activity against prostate cancer. In one aspect, a method of screening for a prostaglandin E synthase 3 (PTGES3) inhibitor for treating prostate cancer is provided, the method comprising: a) contacting PTGES3 with a candidate agent; and b) measuring inhibition of PTGES3 activity by the candidate agent. Screening assays may include determining the effectiveness of candidate PTGES3 inhibitors in reducing proliferation, survival, or androgen receptor abundance of prostate cancer cells.

### BACKGROUND OF THE INVENTION

The concept that a cancerous phenotype can be driven by the activity of a single oncogene has motivated the search for targeted therapeutics directed against individual oncoproteins. In prostate cancer (PCa) we know that the major driver oncogene is the androgen receptor (AR) and that inhibition of AR activity by small molecule drugs prolongs patient survival. Despite this, single agent therapies are rarely curative in cancer, and intrinsic or acquired resistance is a clinical reality thus demonstrating that AR-dependent PCa is driven by more than one oncoprotein and suggesting that combination therapies that deprive AR-dependent PCa will be necessary for curing this disease. It is also likely that upon direct pharmacological inhibition of an oncogene like AR, AR-dependent cancer cells will engage additional genes/pathways to maintain survival and proliferation. Lastly, we know that non-oncogenes additionally are required for maintaining the cancer cell phenotype and that such genetic dependencies are often missed by clinical genomic annotations of the cancer genome.

Thus, there remains a need to identify oncogenes as well as other genes that contribute to the survival of the cancer cell phenotype as potential new therapeutic targets for treating prostate cancer.

### SUMMARY OF THE INVENTION

Methods are provided for screening candidate agents for inhibition of prostaglandin E synthase 3 (PTGES3). Screening assays may include further determining the effectiveness of candidate PTGES3 inhibitors in reducing proliferation, survival, or androgen receptor abundance of prostate cancer cells.

In one aspect, a method of screening for a prostaglandin E synthase 3 (PTGES3) inhibitor for treating prostate cancer is provided, the method comprising: a) contacting PTGES3 with a candidate agent; and b) measuring inhibition of PTGES3 activity by the candidate agent.

In certain embodiments, the method further comprises: contacting a population of prostate cancer cells with the candidate agent if the candidate agent inhibits the PTGES3 activity; and measuring proliferation, survival, or androgen receptor abundance in the population of prostate cancer cells, wherein reduced proliferation, survival, or androgen receptor abundance in the presence of the candidate agent compared to that in a negative control population of prostate cancer cells that are not treated with the candidate agent indicates that the candidate agent has anti-cancer activity.

In some embodiments, the candidate agent inhibits production of prostaglandin E2 from prostaglandin H2. In some embodiments, the candidate agent inhibits PTGES3 protein chaperone activity. In some embodiments, the candidate agent inhibits PTGES3 protein binding to AR or modulation of AR protein activity. In other embodiments, the candidate agent inhibits PTGES3 gene expression. For example, the candidate agent may inhibit enzymatic activity of prostaglandin E synthase, binding to AR, PTGES3 protein chaperone activity, or PTGES3 gene transcription or protein translation.

Candidate agents may include, without limitation, small molecules, peptides, proteins, aptamers, antibodies, antibody mimetics, peptide nucleic acids, inhibitory nucleic acids, or Clustered Regularly Interspaced Short Palindromic Repeats (CRISPR) systems that interfere with PTGES3 biological activity or expression.

In some embodiments, the candidate agent is an antibody that specifically binds to PTGES3, wherein the antibody is selected from the group consisting of a polyclonal antibody, a monoclonal antibody, a chimeric antibody, a humanized antibody, a F(ab) fragment, a F(ab′)2 fragment, a Fᵥ fragment, and a nanobody.

In some embodiments, the candidate agent is an inhibitory nucleic acid selected from the group consisting of a small interfering RNA (siRNA), a microRNA (miRNA), a Piwi-interacting RNA (piRNA), a small nuclear RNA (snRNA), an antisense oligonucleotide, and a peptide nucleic acid.

In some embodiments, the candidate agent is a CRISPR system targeting the PTGES3 gene (e.g., Cas9, Cas12a), a PTGES3 RNA transcript (e.g., Cas13a, Cas13b, or Cas13d), or the epigenome (e.g., dead Cas9 with endonuclease activity deactivated (dCas9)).

In another aspect, a PTGES3 inhibitor identified by the screening methods described herein is provided. In certain embodiments, the PTGES3 inhibitor is provided in a pharmaceutical formulation suitable for administration to a patient. Formulations of interest include, without limitation, formulations for systemic administration, including oral or parenteral administration. In some embodiments, the composition comprises a pharmaceutically acceptable excipient. In some embodiments, the composition further comprises a pharmaceutically acceptable carrier including, without limitation, a cream, emulsion, gel, liposome, nanoparticle, or ointment. Such PTGES3 inhibitors identified by screening, as described herein, may be useful in treating prostate cancer, including, without limitation, prostate adenocarcinoma, small cell prostate cancer, non-small cell prostate cancer, neuroendocrine prostate cancer, or metastatic castration resistant prostate cancer.

### BRIEF DESCRIPTION OF THE DRAWINGS

FIGS. 1A-1B. A CRISPRi screen identifies the regulators of AR. FIG. 1A. Schematic of the fluorescence-activated cell sorting (FACS) based whole genome wide CRISRPi screening in LNcaP^{mNG2-AR} (dCas9-KRAB) cells. FIG. 1B. A volcano plot of screening gene hits (light purple) and negative control (brown) in LNCaP^{mNG2-AR}. Top 5 gene hits upregulating AR are highlighted.

FIGS. 2A-2E. Establishment of the first endogenous AR reporter prostate cancer cell line. FIG. 2A. A diagram of the self-assembling mutated neon green (mNG2) fluorescence protein system. FIG. 2B. The AR tagging design strategy: The 16 amino acid 11^{th} β-strand of the fluorescent protein neon green 2 (mNG2_11) is knocked into the N-terminus of AR by CRISPR-Cas9. When the remainder of mNG2 (mNG2 1-10) is expressed in the same cell, it non-covalently binds to mNG2_11, producing a fluorescent protein. FIG. 2C. Immunoprecipitation (IP) experiments show the mNG2 antibody can pull down mNG2 1-10 and AR; protein levels were detected by western blotting. FIG. 2D. LNCaP^{mNG2-AR} cells were treated with DMSO, R1881 (androgen), R1881 plus Enzalutamide (Enz, anti-androgen), or ARD-61 (AR degrader, PROTAC). Representative confocal microscopic images showed mNG2-AR localization (Green). FIG. 2E. LNCaP and LNCaP^{mNG2-AR} cells were fixed by formaldehyde. ChIP experiments were performed using an AR antibody. Precipitated DNA fragment were used as templates to amplify the PSA enhancer and TMPRSS2 promoter by real-time PCR.

FIGS. 3A-3C. No significant differences of tagged AR mRNA levels, AR protein stability, or AR target gene expression of the AR reporter cell line compared to its parental line. FIG. 3A. LNCaP and LNCaP^{mNG2-AR} cells treated with Actinmycin D for 0, 8 or 16 hours. RNA was collected to measure the AR mRNA levels using real-time PCR. FIG. 3B. LNCaP and LNCaP^{mNG2-AR} cells were treated with protein synthesis inhibitor Cycloheximide (CHX) with or without proteasome inhibitor MG132 for 0, 12, 24 hours. AR protein levels were detected by western blotting. FIG. 3C. Total RNA from LNCaP and LNCaPmNG2-AR cells were collected for RNA-seq. Gene expression values were calculated as In(TPM+0.1). Pearson correlation was calculated comparing all genes (Left; n=19127) or AR pathway gene (Right; n=22;PMID: 17010675) between the two cell lines.

FIGS. 4A-4B. Repression of PTGES3 with sgRNA decreases AR protein levels similar as the canonical AR regulators. FIG. 4A. ^{LNCaPmNG2-AR}(dCas9-KRAB) were infected with sgRNA targeting control, AR, GRHL2, HOXB13, or PTGES3. Median fluorescence intensity was detected by flow cytometry. Relative mNG2-AR levels (%) were normalized to sgGAL4. FIG. 4B. LNCaP(dCas9-KRAB) were infected with sgRNA targeting control, AR, GRHL2, HOXB13, or PTGES3. AR and GAPDH levels were detected by western blotting.

FIGS. 5A-5B. Knockdown of PTGES3 decreases AR protein levels and AR downstream genes. FIG. 5A. C42B, Enzalutamide resistant cell line MR16D, 22RV1, and VCaP cell were treated with siRNA targeting control or PTGES3. AR, PTGES3 and GAPDH levels were detected by western blotting. FIG. 5B. LNCaP(dCas9-KRAB) were infected with sgRNA targeting control or PTGES3. Total RNA was collected for RNA-seq. The expression of AR downstream genes is shown in the heat map.

FIGS. 6A-6F. Knockdown of PTGES3 decrease AR positive prostate cancer cells and LNCaP tumor xenograft growth. C42B (FIG. 6A), 22RV1 (FIG. 6B) VCaP (FIG. 6C), Enzalutamide (Enz) resistant cell line MR16D culturing with 5 μM Enz (FIG. 6D), PC3/DU145 AR negative prostate cancer cell lines (FIG. 6E) were treated with siRNA targeting control or PTGES3. MTS assays were measured the relative cell growth rates to day 0. FIG. 6F. Mice bearing LNCaP (TET-ON dCas9-KRAB) stably express sgRNA target control or sgPTGES3 were treated with doxycycline when tumor reached −200 mm³. Tumor volumes were measured weekly.

FIG. 7. PTGES3 is not required for cell proliferation or survival in most cell lines. A histogram of gene essentiality scores from the Cancer Dependency map showing the distribution of PTGES3 essentiality in 739 cell lines. PTGES3 is considered essential for cell proliferation or survival in 41 cell lines (−5% of all cell lines) with scores less than −0.5. The median phenotype for a common essential gene is −1 shown by a read line. This result indicates PTGES3 is not required for cell growth or survival in most cell types.

FIGS. 8A-8B. Overexpression of PTGES3 confers resistance to AR degrader ARD-61. FIG. 8A. LNCaP (TET-ON PTGES3) cells were treated with increasing dose of ARD-61 plus DMSO or 100 ng/ml doxycycline. AR, GAPDH, and Flag-PTGES3 levels were detected by western blotting. FIG. 8B. LNCaP (TET-ON PTGES3) cells treated with increasing dose of ARD-61 plus DMSO or 100 ng/ml doxycycline for 5 days. Cell viability were measured by WST1 and normalized over control.

FIGS. 9A-9B. PTGES3 has a nuclear localization and binds to AR. FIG. 9A. Nuclear (N) and cytosol (C) extracts from LNCaP and C42B cells were immunoblotted with indicated antibodies. FIG. 9B. IP experiments were performed using PTGES3 antibody. AR and KAT2A protein levels were detected by western blotting.

FIG. 10. PTGES3 indirectly binds to Androgen Response Elements (ARE). LNCaP cells were fixed sequentially with EGS and formaldehyde. Dual cross-linking ChIP experiments were performed using a PTGES3 antibody. Precipitated DNA was used as a template to amplify the indicated AREs by real-time PCR.

### DETAILED DESCRIPTION

Methods and compositions are provided for screening candidate agents for inhibition of prostaglandin E synthase 3 (PTGES3) and anti-cancer activity against prostate cancer. Screening assays may include determining the effectiveness of candidate PTGES3 inhibitors in reducing proliferation, survival, or androgen receptor abundance of prostate cancer cells. PTGES3 inhibitors may include, without limitation, small molecules, chimeric proteins/peptides, bioactive polypeptides, antibodies, aptamers, and inhibitory nucleic acids, e.g. RNAi and antisense nucleic acids that interfere with PTGES3, etc.

Before the methods and compositions are provided for screening candidate agents for inhibition of PTGES3 and anti-cancer activity against prostate cancer are further described, it is to be understood that this invention is not limited to a particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited.

It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

As used herein the singular forms “a”, “an”, and “the” include plural referents unless the context clearly dictates otherwise. Thus, for example, reference to “an enzyme” includes a plurality of such enzymes and reference to “the inhibitor” includes reference to one or more inhibitors and equivalents thereof, e.g., antagonists, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The term “about,” particularly in reference to a given quantity, is meant to encompass deviations of plus or minus five percent.

The term “administering” is intended to include routes of administration which allow the agent to perform its intended function of inhibiting biological activity or expression of PTGES3 and/or growth/proliferation of prostate cancer cells. Examples of routes of administration which can be used include injection (subcutaneous, intravenous, parenterally, intraperitoneally, intrathecal, etc.), oral, inhalation, and transdermal. The injection can be bolus injections or can be continuous infusion. Depending on the route of administration, the agent can be coated with or disposed in a selected material to protect it from natural conditions which may detrimentally affect its ability to perform its intended function. The agent may be administered alone, or in conjunction with a pharmaceutically acceptable carrier. Further, the agent may be coadministered with a pharmaceutically acceptable carrier. The agent also may be administered as a prodrug, which is converted to its active form in vivo.

As used herein, the term “determining” refers to both quantitative and qualitative determinations and as such, the term “determining” is used interchangeably herein with “assaying,” “measuring,” and the like.

“Substantially purified” generally refers to isolation of a substance (e.g., compound, polynucleotide, protein, polypeptide, antibody, aptamer, PTGES3 inhibitor) such that the substance comprises the majority percent of the sample in which it resides. Typically in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

By “isolated” is meant, when referring to a polypeptide or peptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

A peptide is said to “interact” with a protein if it binds specifically (e.g., in a lock-and-key type mechanism), non-specifically or in some combination of specific and non-specific binding. A first peptide “interacts preferentially” with a protein if it binds (non-specifically and/or specifically) to the protein with greater affinity and/or greater specificity than it binds to other proteins (e.g., binds to PTGES3 to a greater degree than to other proteins). The term “affinity” refers to the strength of binding and can be expressed quantitatively as a dissociation constant (Kd). It is to be understood that specific binding does not necessarily require interaction between specific amino acid residues and/or motifs of each peptide. For example, in certain embodiments, a peptide interacts preferentially with PTGES3 but, nonetheless, may be capable of binding other polypeptides at a weak, yet detectable, level (e.g., 10% or less of the binding shown to the polypeptide of interest). Typically, weak binding, or background binding, is readily discernible from the preferential interaction with the compound or polypeptide of interest, e.g., by use of appropriate controls.

A PTGES3 polynucleotide, nucleic acid, oligonucleotide, protein, polypeptide, or peptide refers to a molecule derived from any source. The molecule need not be physically derived from an organism but may be synthetically or recombinantly produced. A number of PTGES3 nucleic acid and protein sequences are known. Representative PTGES3 sequences are presented in SEQ ID NO:1 and SEQ ID NO:2 and additional representative sequences are listed in the National Center for Biotechnology Information (NCBI) database, including sequences for various isoforms of PTGES3. See, for example, NCBI entries: Accession Nos. NM_001282604, XM_005268576, XM_006719199, XM_011537774, XM_011537773, XM_017018716, NR_104219, NM_001282603, NM_001282603, NM_001282601, NM_001282605, NM_006601, XP_016874205, XP_011536076, XP_011536075, XP_006719262, XP_005268633, NP_001269533, NP_001269534, NP_001269530, NP_001269532, NP_001269531, and NP_006592; all of which sequences (as entered by the date of filing of this application) are herein incorporated by reference.

The term “PTGES3 inhibitor” as used herein refers to any molecule (e.g., small molecule inhibitor, protein, polypeptide, peptide, fusion protein, inhibitory nucleic acid (e.g., siRNA, miRNA, antisense nucleic acid), peptide nucleic acid, antibody, antibody mimetic, aptamer) or CRISPR system targeting the PTGES3 gene (e.g., Cas9, Cas12a), PTGES3 RNA transcripts (e.g., Cas13), or epigenome (e.g., dCas9 fusion protein) that inhibits PTGES3 biological activity (e.g., enzymatic activity or chaperone activity), and/or PTGES3 expression (e.g., transcription or translation). The PTGES3 inhibitor may inhibit one or more PTGES3 isoforms. In some embodiments, the PTGES3 inhibitor selectively inhibits one PTGES3 isoform. Inhibition may be complete or partial (i.e., all activity, some activity, or most activity is blocked by an inhibitor). For example, an inhibitor may reduce the activity of PTGES3 by 10%, 20%, 30%, 40%, 50%, 60%, 70%, 80%, 90%, 100%, or any amount in between as compared to native or control levels.

An “effective amount” of a PTGES3 inhibitor (e.g., small molecule inhibitor, protein, polypeptide, peptide, fusion protein, inhibitory nucleic acid (e.g., siRNA, miRNA, antisense nucleic acid, or peptide nucleic acid), antibody, antibody mimetic, or aptamer) or CRISPR system targeting the PTGES3 gene (e.g., Cas9, Cas12a), RNA (Cas13), or epigenome (dCas9 fusion protein) is an amount sufficient to inhibit the biological activity of PTGES3, for example, by inhibiting prostaglandin E synthase 3 enzymatic activity or chaperone activity or interfering with PTGES3 gene expression (e.g., transcription or translation). An effective amount can be administered in one or more administrations, applications, or dosages.

The terms “tumor,” “cancer” and “neoplasia” are used interchangeably and refer to a cell or population of cells whose growth, proliferation or survival is greater than growth, proliferation or survival of a normal counterpart cell, e.g. a cell proliferative, hyperproliferative or differentiative disorder. Typically, the growth is uncontrolled. The term “malignancy” refers to invasion of nearby tissue. The term “metastasis” or a secondary, recurring or recurrent tumor, cancer or neoplasia refers to spread or dissemination of a tumor, cancer or neoplasia to other sites, locations or regions within the subject, in which the sites, locations or regions are distinct from the primary tumor or cancer. Neoplasia, tumors and cancers include benign, malignant, metastatic and non-metastatic types, and include any stage (I, II, Ill, IV or V) or grade (G1, G2, G3, etc.) of neoplasia, tumor, or cancer, or a neoplasia, tumor, cancer or metastasis that is progressing, worsening, stabilized or in remission. In particular, the terms “tumor,” “cancer” and “neoplasia” include carcinomas, such as squamous cell carcinoma, adenocarcinoma, adenosquamous carcinoma, anaplastic carcinoma, large cell carcinoma, and small cell carcinoma.

“Prostate cancer” refers to any type of prostate cancer of any stage or grade, including, without limitation, prostate adenocarcinoma, small cell prostate cancer, non-small cell prostate cancer, neuroendocrine prostate cancer, or metastatic castration resistant prostate cancer.

By “anti-tumor activity” or “anti-cancer activity” is intended a reduction in the rate of cell proliferation, and hence a decline in growth rate of an existing tumor or in a tumor that arises during therapy, and/or destruction of existing neoplastic (tumor) cells or newly formed neoplastic cells, and hence a decrease in the overall size of a tumor during therapy. Such activity can be assessed using animal models.

By “therapeutically effective dose or amount” of a PTGES3 inhibitor is intended an amount that, when administered as described herein, brings about a positive therapeutic response in treatment of prostate cancer, such as an amount having anti-tumor activity. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein.

The term “tumor response” as used herein means a reduction or elimination of all measurable lesions. The criteria for tumor response are based on the WHO Reporting Criteria [WHO Offset Publication, 48-World Health Organization, Geneva, Switzerland, (1979)]. Ideally, all uni- or bidimensionally measurable lesions should be measured at each assessment. When multiple lesions are present in any organ, such measurements may not be possible and, under such circumstances, up to 6 representative lesions should be selected, if available.

The term “complete response” (CR) as used herein means a complete disappearance of all clinically detectable malignant disease, determined by 2 assessments at least 4 weeks apart.

The term “partial response” (PR) as used herein means a 50% or greater reduction from baseline in the sum of the products of the longest perpendicular diameters of all measurable disease without progression of evaluable disease and without evidence of any new lesions as determined by at least two consecutive assessments at least four weeks apart. Assessments should show a partial decrease in the size of lytic lesions, recalcifications of lytic lesions, or decreased density of blastic lesions.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

By “subject” is meant any member of the subphylum chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like.

The term “antibody” encompasses polyclonal antibodies, monoclonal antibodies as well as hybrid antibodies, altered antibodies, chimeric antibodies, and humanized antibodies. The term antibody includes: hybrid (chimeric) antibody molecules (see, for example, Winter et al. (1991) Nature 349:293-299; and U.S. Pat. No. 4,816,567); F(ab′)₂ and F(ab) fragments; Fᵥ molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (scFv) (see, e.g., Huston et al. (1988) Proc Natl Acad Sci USA 85:5879-5883); nanobodies or single-domain antibodies (sdAb) (see, e.g., Wang et al. (2016) Int J Nanomedicine 11:3287-3303, Vincke et al. (2012) Methods Mol Biol 911:15-26; dimeric and trimeric antibody fragment constructs; minibodies (see, e.g., Pack et al. (1992) Biochem 31:1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

The phrase “specifically (or selectively) binds” with reference to binding of an antibody to an antigen (e.g., PTGES3) refers to a binding reaction that is determinative of the presence of the antigen in a heterogeneous population of proteins and other biologics. Thus, under designated immunoassay conditions, the specified antibodies bind to a particular antigen at least two times the background and do not substantially bind in a significant amount to other antigens present in the sample. Specific binding to an antigen under such conditions may require an antibody that is selected for its specificity for a particular antigen. For example, antibodies raised to an antigen from specific species such as rat, mouse, or human can be selected to obtain only those antibodies that are specifically immunoreactive with the antigen and not with other proteins, except for polymorphic variants and alleles. This selection may be achieved by subtracting out antibodies that cross-react with molecules from other species. A variety of immunoassay formats may be used to select antibodies specifically immunoreactive with a particular antigen. For example, solid-phase ELISA immunoassays are routinely used to select antibodies specifically immunoreactive with a protein (see, e.g., Harlow & Lane. Antibodies, A Laboratory Manual (1988), for a description of immunoassay formats and conditions that can be used to determine specific immunoreactivity). Typically, a specific or selective reaction will be at least twice background signal or noise and more typically more than 10 to 100 times background.

The terms “microRNA,” “miRNA,” and MiR” are interchangeable and refer to endogenous or artificial non-coding RNAs that are capable of regulating gene expression. It is believed that miRNAs function via RNA interference. When used herein in the context of inactivation, the use of the term microRNAs is intended to include also long non-coding RNAs, piRNAs, siRNAs, and the like. Endogenous (e.g., naturally occurring) miRNAs are typically expressed from RNA polymerase II promoters and are generated from a larger transcript.

The terms “siRNA” and “short interfering RNA” are interchangeable and refer to single-stranded or double-stranded RNA molecules that are capable of inducing RNA interference. SiRNA molecules typically have a duplex region that is between 18 and 30 base pairs in length.

The terms “piRNA” and “Piwi-interacting RNA” are interchangeable and refer to a class of small RNAs involved in gene silencing. PiRNA molecules typically are between 26 and 31 nucleotides in length.

The terms “snRNA” and “small nuclear RNA” are interchangeable and refer to a class of small RNAs involved in a variety of processes including RNA splicing and regulation of transcription factors. The subclass of small nucleolar RNAs (snoRNAs) is also included. The term is also intended to include artificial snRNAs, such as antisense derivatives of snRNAs comprising antisense sequences directed against the PTGES3 gene.

The term “antisense”, as used herein, refers to any composition containing nucleotide sequences which are complementary to a specific DNA or RNA sequence. The term “antisense strand” is used in reference to a nucleic acid strand that is complementary to the “sense” strand. Antisense molecules include peptide nucleic acids and may be produced by any method including synthesis or transcription. Once introduced into a cell, the complementary nucleotides combine with natural sequences produced by the cell to form duplexes and block either transcription or translation. The designation “negative” is sometimes used in reference to the antisense strand, and “positive” is sometimes used in reference to the sense strand.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms will be used interchangeably. Thus, these terms include, for example, 3′-deoxy-2′,5′-DNA, oligodeoxyribonucleotide N3′ P5′ phosphoramidates, 2′-O-alkyl-substituted RNA, double-and single-stranded DNA, as well as double- and single-stranded RNA, microRNA, DNA:RNA hybrids, and hybrids between PNAs and DNA or RNA, and also include known types of modifications, for example, labels which are known in the art, methylation, “caps,” substitution of one or more of the naturally occurring nucleotides with an analog (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide. The term also includes locked nucleic acids (e.g., comprising a ribonucleotide that has a methylene bridge between the 2′-oxygen atom and the 4′-carbon atom). See, for example, Kurreck et al. (2002) Nucleic Acids Res. 30: 1911-1918; Elayadi et al. (2001) Curr. Opinion Invest. Drugs 2: 558-561; Orum et al. (2001) Curr. Opinion Mol. Ther. 3: 239-243; Koshkin et al. (1998) Tetrahedron 54: 3607-3630; Obika et al. (1998) Tetrahedron Lett. 39: 5401-5404.

The term “homologous region” refers to a region of a nucleic acid with homology to another nucleic acid region. Thus, whether a “homologous region” is present in a nucleic acid molecule is determined with reference to another nucleic acid region in the same or a different molecule. Further, since a nucleic acid is often double-stranded, the term “homologous, region,” as used herein, refers to the ability of nucleic acid molecules to hybridize to each other. For example, a single-stranded nucleic acid molecule can have two homologous regions which are capable of hybridizing to each other. Thus, the term “homologous region” includes nucleic acid segments with complementary sequence. Homologous regions may vary in length, but will typically be between 4 and 40 nucleotides (e.g., from about 4 to about 40, from about 5 to about 40, from about 5 to about 35, from about 5 to about 30, from about 5 to about 20, from about 6 to about 30, from about 6 to about 25, from about 6 to about 15, from about 7 to about 18, from about 8 to about 20, from about 8 to about 15, etc.).

The term “complementary” and “complementarity” are interchangeable and refer to the ability of polynucleotides to form base pairs with one another. Base pairs are typically formed by hydrogen bonds between nucleotide units in antiparallel polynucleotide strands or regions. Complementary polynucleotide strands or regions can base pair in the Watson-Crick manner (e.g., A to T, A to U, C to G). 100% complementary refers to the situation in which each nucleotide unit of one polynucleotide strand or region can hydrogen bond with each nucleotide unit of a second polynucleotide strand or region. Less than perfect complementarity refers to the situation in which some, but not all, nucleotide units of two strands or two regions can hydrogen bond with each other and can be expressed as a percentage.

A “target site” or “target sequence” for an inhibitory nucleic acid is the nucleic acid sequence recognized (i.e., sufficiently complementary for hybridization) by an antisense oligonucleotide or inhibitory RNA molecule.

The term “transfection” is used to refer to the uptake of foreign DNA or RNA by a cell. A cell has been “transfected” when exogenous DNA or RNA has been introduced inside the cell membrane. A number of transfection techniques are generally known in the art. See, e.g., Graham et al. (1973) Virology, 52:456, Sambrook et al. (2001) Molecular Cloning, a laboratory manual, 3rd edition, Cold Spring Harbor Laboratories, New York, Davis et al. (1995) Basic Methods in Molecular Biology, 2nd edition, McGraw-Hill, and Chu et al. (1981) Gene 13:197. Such techniques can be used to introduce one or more exogenous DNA or RNA moieties into suitable host cells. The term refers to both stable and transient uptake of the genetic material, and includes uptake, for example, of microRNA, siRNA, piRNA, IncRNA, or antisense nucleic acids.

A “CRISPR system” refers collectively to transcripts and other elements involved in the expression of or directing the activity of CRISPR-associated (“Cas”) genes. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence.

The term “Cas9” as used herein encompasses type II clustered regularly interspaced short palindromic repeats (CRISPR) system Cas9 endonucleases from any species, and also includes biologically active fragments, variants, analogs, and derivatives thereof that retain Cas9 endonuclease activity (i.e., catalyze site-directed cleavage of DNA to generate double-strand breaks).

A Cas9 endonuclease binds to and cleaves DNA at a site comprising a sequence complementary to its bound guide RNA (gRNA). For purposes of Cas9 targeting, a gRNA may comprise a sequence “complementary” to a target sequence (e.g., major or minor allele), capable of sufficient base-pairing to form a duplex (i.e., the gRNA hybridizes with the target sequence). Additionally, the gRNA may comprise a sequence complementary to a PAM sequence, wherein the gRNA also hybridizes with the PAM sequence in a target DNA.

By “selectively binds” with reference to a guide RNA is meant that the guide RNA binds preferentially to a target sequence of interest or binds with greater affinity to the target sequence than to other genomic sequences. For example, a gRNA will bind to a substantially complementary sequence and not to unrelated sequences. A gRNA that “selectively binds” to a particular allele, such as a particular mutant allele (e.g., allele comprising a substitution, insertion, or deletion), denotes a gRNA that binds preferentially to the particular target allele, but to a lesser extent to a wild-type allele or other sequences. A gRNA that selectively binds to a particular target DNA sequence will selectively direct binding of Cas9 to a substantially complementary sequence at the target site and not to unrelated sequences.

The term “donor polynucleotide” refers to a polynucleotide that provides a sequence of an intended edit to be integrated into the genome at a target locus by homology directed repair (HDR).

A “target site” or “target sequence” is the nucleic acid sequence recognized (i.e., sufficiently complementary for hybridization) by a guide RNA (gRNA) or a homology arm of a donor polynucleotide. The target site may be allele-specific (e.g., a major or minor allele).

By “homology arm” is meant a portion of a donor polynucleotide that is responsible for targeting the donor polynucleotide to the genomic sequence to be edited in a cell. The donor polynucleotide typically comprises a 5′ homology arm that hybridizes to a 5′ genomic target sequence and a 3′ homology arm that hybridizes to a 3′ genomic target sequence flanking a nucleotide sequence comprising the intended edit to the genomic DNA. The homology arms are referred to herein as 5′ and 3′ (i.e., upstream and downstream) homology arms, which relates to the relative position of the homology arms to the nucleotide sequence comprising the intended edit within the donor polynucleotide. The 5′ and 3′ homology arms hybridize to regions within the target locus in the genomic DNA to be modified, which are referred to herein as the “5′ target sequence” and “3′ target sequence,” respectively. The nucleotide sequence comprising the intended edit is integrated into the genomic DNA by HDR or recombineering at the genomic target locus recognized (i.e., sufficiently complementary for hybridization) by the 5′ and 3′ homology arms.

“Administering” a nucleic acid, such as an inhibitory or regulatory nucleic acid (e.g., microRNA, siRNA, piRNA, snRNA, antisense nucleic acid, or IncRNA), or a CRISPR system (expressing, e.g., a donor polynucleotide, guide RNA, Cas protein (e.g., Cas9, Cas12a, Cas12d, Cas13, or dCas9)) to a cell comprises transducing, transfecting, electroporating, translocating, fusing, phagocytosing, shooting or ballistic methods, etc., i.e., any means by which a nucleic acid can be transported across a cell membrane.

### Screening for PTGES3 Inhibitors

The inventors have discovered in genome-wide screening that PTGES3 plays important roles in cell proliferation, survival, and AR abundance in prostate cancer (see Examples). Therefore, inhibitors of PTGES3 may be useful in treating prostate cancer. Accordingly, screening methods for identifying candidate agents that inhibit PTGES3 activity and have anti-tumor activity against prostate cancer are provided.

A variety of assays may be used for this purpose, and in many embodiments, a candidate agent will be tested in different assays to confirm inhibitory capability as well as efficacy in treating prostate cancer. For example, biochemical assays may determine the ability of an agent to inhibit biological activity of PTGES3 (e.g., enzymatic activity and/or chaperone activity). In addition, cell-based assays may be used, for example, for testing for growth, proliferation, or AR abundance of prostate cancer cells in the absence or presence of a candidate agent.

A “PTGES3 inhibitor” can be any molecule including, without limitation, a small molecule inhibitor, protein, polypeptide, peptide, fusion protein, nucleic acid, oligonucleotide, peptide nucleic acid, antibody or fragment thereof, antibody mimetic, aptamer, or a CRISPR system targeting the PTGES3 gene (e.g., Cas9, Cas12a), RNA transcripts (e.g., Cas13), or epigenome (e.g., dCas9 fusion protein) that inhibits PTGES3 activity and/or PTGES3 expression (e.g., transcription or translation). Inhibition may be complete or partial (i.e., all activity, some activity, or most activity is blocked by an inhibitor). For example, an inhibitor may reduce the activity of PTGES3 or reduce PTGES3 mRNA or protein levels by 10%, 20%, 30%, 40%, 50%, 60%, 70%, 80%, 90%, 100%, or any amount in between as compared to native or control levels. The PTGES3 inhibitor may inhibit one or more isoforms of PTGES3. In some embodiments, the PTGES3 inhibitor selectively inhibits one PTGES3 isoform.

PTGES3 acts in both the cytosol and nucleus to support AR function. PTGES3 catalyzes the oxidoreduction of prostaglandin endoperoxide H2 (PGH2) to prostaglandin E2 (PGE2). In addition, PTGES3 has molecular chaperone activity. PTGES3 indirectly binds to androgen response elements (ARE) and may play a role in AR transcriptional regulation. PTGES3 also facilitates HIF alpha protein hydroxylation through interaction with EGLN1/PHD2, leading to recruitment of EGLN1/PHD2 to the HSP90 pathway. In some embodiments, a PTGES3 inhibitor reduces biological activity of PTGES3 by 10%, 20%, 30%, 40%, 50%, 60%, 70%, 80%, 90%, 100%, or any amount in between as compared to native or control levels. In some embodiments, a PTGES3 inhibitor reduces enzymatic activity and/or chaperone activity and/or regulatory activity of PTGES3.

For purposes of the assay methods, PTGES3 may be provided as an isolated protein. Alternatively, the PTGES3 protein can be present in the context of a cell. Any convenient format may be used for the assay, e.g. wells, plates, flasks, etc., preferably a high throughput format, such as multi-well plates. A test agent of interest is added to the reaction mixture with the PTGES3 protein, for example in the presence of the PGH2 substrate, and the effect of the agent on PTGES3 activity is determined.

Inhibitors can be identified by contacting PTGES3 with a candidate agent; and measuring inhibition of PTGES3 biological activity (e.g., prostaglandin E synthase 3 enzymatic activity and/or chaperone activity) by the candidate agent. For example, inhibition of enzymatic activity can be assayed by detecting a decreased rate of production of PGE2 from PGH2 in the presence of the candidate agent compared to that in the absence of the candidate agent. The assay can be performed, for example, in a buffered solution containing prostaglandin E synthase 3, the candidate agent, and the substrate PGH2 at about pH 8.0. The reaction can be terminated by the addition of FeCl₂. The PGE2 product can be detected by methods known in the art such as by 9-anthryldiazomethane -reversed-phase high performance liquid chromatography (see, e.g., Kurosawa et al. (1990) Ann Allergy. 64(5):464-70. Naraba et al. (1998) J. Immunol. 160(6):2974-82; Matsumoto et al. (1997) Biochem. Biophys. Res. Commun. 230:110; herein incorporated by reference in their entireties).

Assays may further include suitable controls (e.g., a sample comprising the PTGES3 protein in the absence of the test agent). Generally, a plurality of assay mixtures is run in parallel with different agent concentrations to obtain a differential response to the various concentrations. Typically, one of these concentrations serves as a negative control, i.e. at zero concentration or below the level of detection.

A variety of other reagents may be included in the screening assay. These include reagents like salts, neutral proteins, e.g. albumin, detergents, etc., including agents that are used to facilitate optimal binding activity and/or reduce non-specific or background activity. Reagents that improve the efficiency of the assay, such as protease inhibitors, nuclease inhibitors, anti-microbial agents, etc. may be used. The components of the assay mixture are added in any order that provides for the requisite activity. Incubations are performed at any suitable temperature, typically between 4° C. and 40° C. Incubation periods are selected for optimum activity but may also be optimized to facilitate rapid high-throughput screening. In some embodiments, between 0.1 hour and 1 hour, between 1 hour and 2 hours, or between 2 hours and 4 hours, will be sufficient.

A variety of different test agents may be screened. Candidate agents encompass numerous chemical classes, e.g., small organic compounds having a molecular weight of more than 50 daltons and less than about 10,000 daltons, less than about 5,000 daltons, or less than about 2,500 daltons. Test agents can comprise functional groups necessary for structural interaction with proteins, e.g., hydrogen bonding, and can include at least an amine, carbonyl, hydroxyl or carboxyl group, or at least two of the functional chemical groups. The test agents can comprise cyclical carbon or heterocyclic structures and/or aromatic or polyaromatic structures substituted with one or more of the above functional groups. Test agents are also found among biomolecules including peptides, saccharides, fatty acids, steroids, purines, pyrimidines, derivatives, structural analogs or combinations thereof.

Test agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Moreover, screening may be directed to known pharmacologically active compounds and chemical analogs thereof, or to new agents with unknown properties such as those created through rational drug design.

In some embodiments, test agents are synthetic compounds. A number of techniques are available for the random and directed synthesis of a wide variety of organic compounds and biomolecules, including expression of randomized oligonucleotides. See for example WO 94/24314, hereby expressly incorporated by reference, which discusses methods for generating new compounds, including random chemistry methods as well as enzymatic methods.

In another embodiment, the test agents are provided as libraries of natural compounds in the form of bacterial, fungal, plant and animal extracts that are available or readily produced. Additionally, natural or synthetically produced libraries and compounds are readily modified through conventional chemical, physical and biochemical means. Known pharmacological agents may be subjected to directed or random chemical modifications, including enzymatic modifications, to produce structural analogs.

In some embodiments, the test agents are organic moieties. In this embodiment, test agents are synthesized from a series of substrates that can be chemically modified. “Chemically modified” herein includes traditional chemical reactions as well as enzymatic reactions. These substrates generally include, but are not limited to, alkyl groups (including alkanes, alkenes, alkynes and heteroalkyl), aryl groups (including arenes and heteroaryl), alcohols, ethers, amines, aldehydes, ketones, acids, esters, amides, cyclic compounds, heterocyclic compounds (including purines, pyrimidines, benzodiazepins, beta-lactams, tetracylines, cephalosporins, and carbohydrates), steroids (including estrogens, androgens, cortisone, ecodysone, etc.), alkaloids (including ergots, vinca, curare, pyrollizdine, and mitomycines), organometallic compounds, hetero-atom bearing compounds, amino acids, and nucleosides. Chemical (including enzymatic) reactions may be done on the moieties to form new substrates or candidate agents which can then be tested using the present invention.

In some embodiments test agents are assessed for any cytotoxic activity it may exhibit toward a living eukaryotic cell, using well-known assays, such as trypan blue dye exclusion, an MTT (3-(4,5-dimethylthiazol-2-yl)-2,5-diphenyl-2 H-tetrazolium bromide) assay, and the like. Agents that do not exhibit significant cytotoxic activity are considered candidate agents.

In some embodiments, the test agent is an antibody that specifically binds to and inhibits biological activity of PTGES3. Any type of antibody may be screened for the ability to inhibit PTGES3 by the methods described herein, including polyclonal antibodies, monoclonal antibodies, hybrid antibodies, altered antibodies, chimeric antibodies and, humanized antibodies, as well as: hybrid (chimeric) antibody molecules (see, for example, Winter et al. (1991) Nature 349:293-299; and U.S. Pat. No. 4,816,567); F(ab′)₂ and F(ab) fragments; Fᵥ molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (sFv) (see, e.g., Huston et al. (1988) Proc Natl Acad Sci USA 85:5879-5883); nanobodies or single-domain antibodies (sdAb) (see, e.g., Wang et al. (2016) Int J Nanomedicine 11:3287-3303, Vincke et al. (2012) Methods Mol Biol 911:15-26; dimeric and trimeric antibody fragment constructs; minibodies (see, e.g., Pack et al. (1992) Biochem 31:1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

In other embodiments, the test agent is an aptamer that specifically binds to and inhibits biological activity of PTGES3. Aptamers may be isolated from a combinatorial library and improved by directed mutation or repeated rounds of mutagenesis and selection. For a description of methods of producing aptamers, see, e.g., Aptamers: Tools for Nanotherapy and Molecular Imaging (R. N. Veedu ed., Pan Stanford, 2016), Nucleic Acid and Peptide Aptamers: Methods and Protocols (Methods in Molecular Biology, G. Mayer ed., Humana Press, 2009), Aptamers Selected by Cell-SELEX for Theranostics (W. Tan, X. Fang eds., Springer, 2015), Cox et al. (2001) Bioorg. Med. Chem. 9(10):2525-2531; Cox et al. (2002) Nucleic Acids Res. 30(20): e108, Kenan et al. (1999) Methods Mol. Biol. 118:217-231; Platella et al. (2016) Biochim. Biophys. Acta Nov 16 pii: S0304-4165(16)30447-0, and Lyu et al. (2016) Theranostics 6(9):1440-1452; herein incorporated by reference in their entireties.

In yet other embodiments, the test agent is an antibody mimetic that specifically binds to and inhibits biological activity of PTGES3. Any type of antibody mimetic may be used as an inhibitor, including, but not limited to, affibody molecules (Nygren (2008) FEBS J. 275 (11):2668-2676), affilins (Ebersbach et al. (2007) J. Mol. Biol. 372 (1):172-185), affimers (Johnson et al. (2012) Anal. Chem. 84 (15):6553-6560), affitins (Krehenbrink et al. (2008) J. Mol. Biol. 383 (5):1058-1068), alphabodies (Desmet et al. (2014) Nature Communications 5:5237), anticalins (Skerra (2008) FEBS J. 275 (11):2677-2683), avimers (Silverman et al. (2005) Nat. Biotechnol. 23 (12):1556-1561), darpins (Stumpp et al. (2008) Drug Discov. Today 13 (15-16):695-701), fynomers (Grabulovski et al. (2007) J. Biol. Chem. 282 (5):3196-3204), and monobodies (Koide et al. (2007) Methods Mol. Biol. 352:95-109).

### Screening for Inhibitors of PTGES3 Gene Expression

Alternatively, a PTGES3 inhibitor may reduce PTGES3 gene expression (e.g., transcription or translation). Candidate agents are identified by contacting a cell with a candidate compound and measuring the expression of PTGES3, as determined by e.g., mRNA or polypeptide levels. The level of expression of PTGES3 mRNA or protein in the presence of the candidate compound is compared to the level of expression of PTGES3 mRNA or protein in the absence of the candidate compound. The candidate compound can then be identified based on this comparison. For example, when expression of PTGES3 mRNA or protein in cells is decreased (statistically significantly less) in the presence of the candidate compound than in its absence, the candidate compound is identified as an agent that inhibits PTGES3 expression. Alternatively, when expression of PTGES3 mRNA or protein is the same or increased (statistically significantly more) in the presence of the candidate compound than in its absence, the candidate compound is likely not an agent that inhibits PTGES3 expression or activity.

In some embodiments, an inhibitor of PTGES3 gene expression reduces PTGES3 mRNA or protein levels by 10%, 20%, 30%, 40%, 50%, 60%, 70%, 80%, 90%, 100%, or any amount in between as compared to native or control levels. Inhibitors of PTGES3 gene expression can include, but are not limited to, antisense oligonucleotides, inhibitory RNA molecules, such as miRNAs, siRNAs, piRNAs, and snRNAs, peptide nucleic acids, small molecule inhibitors, and CRISPR systems designed for genome, RNA transcript, or epigenome editing. Various types of inhibitors for inhibiting nucleic acid function are well known in the art. See e.g., International patent application WO/2012/018881; U.S. patent application 2011/0251261; U.S. Pat. No. 6,713,457; Kole et al. (2012) Nat. Rev. Drug Discov. 11(2):125-40; Sanghvi (2011) Curr. Protoc. Nucleic Acid Chem. Chapter 4:Unit 4.1.1-22; herein incorporated by reference in their entireties.

Inhibitors can be single stranded or double stranded polynucleotides and may contain one or more chemical modifications, such as, but not limited to, locked nucleic acids, peptide nucleic acids, sugar modifications, such as 2′-O-alkyl (e.g., 2′-O-methyl, 2′-O-methoxyethyl), 2′-fluoro, and 4′-thio modifications, and backbone modifications, such as one or more phosphorothioate, morpholino, or phosphonocarboxylate linkages. In addition, inhibitory RNA molecules may have a “tail” covalently attached to their 3′- and/or 5′-end, which may be used to stabilize the RNA inhibitory molecule or enhance cellular uptake. Such tails include, but are not limited to, intercalating groups, various kinds of reporter groups, and lipophilic groups attached to the 3′ or 5′ ends of the RNA molecules. In certain embodiments, the RNA inhibitory molecule is conjugated to cholesterol or acridine. See, for example, the following for descriptions of syntheses of 3′-cholesterol or 3′-acridine modified oligonucleotides: Gamper, H. B., Reed, M. W., Cox, T., Virosco, J. S., Adams, A. D., Gall, A., Scholler, J. K., and Meyer, R. B. (1993) Facile Preparation and Exonuclease Stability of 3′-Modified Oligodeoxynucleotides. Nucleic Acids Res. 21 145-150; and Reed, M. W., Adams, A. D., Nelson, J. S., and Meyer, R. B., Jr. (1991) Acridine and Cholesterol-Derivatized Solid Supports for Improved Synthesis of 3′-Modified Oligonucleotides. Bioconjugate Chem. 2 217-225 (1993); herein incorporated by reference in their entireties. Additional lipophilic moieties that can be used, include, but are not limited to, oleyl, retinyl, and cholesteryl residues, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O₃-(oleoyl)lithocholic acid, O₃-(oleoyl)cholenic acid, dimethoxytrityl, or phenoxazine. Additional compounds, and methods of use, are set out in US Patent Publication Nos. 2010/0076056, 2009/0247608 and 2009/0131360; herein incorporated by reference in their entireties.

In one embodiment, inhibition of PTGES3 function may be achieved by administering antisense oligonucleotides targeting the PTGES3 gene. The antisense oligonucleotides may be ribonucleotides or deoxyribonucleotides. Preferably, the antisense oligonucleotides have at least one chemical modification. Antisense oligonucleotides may be comprised of one or more “locked nucleic acids”. “Locked nucleic acids” (LNAs) are modified ribonucleotides that contain an extra bridge between the 2′ and 4′ carbons of the ribose sugar moiety resulting in a “locked” conformation that confers enhanced thermal stability to oligonucleotides containing the LNAs. Alternatively, the antisense oligonucleotides may comprise peptide nucleic acids (PNAs), which contain a peptide-based backbone rather than a sugar-phosphate backbone. The antisense oligonucleotides may contain one or more chemical modifications, including, but are not limited to, sugar modifications, such as 2′-O-alkyl (e.g. 2′-O-methyl, 2′-O-methoxyethyl), 2′-fluoro, and 4′ thio modifications, and backbone modifications, such as one or more phosphorothioate, morpholino, or phosphonocarboxylate linkages (see, for example, U.S. Pat. Nos. 6,693,187 and 7,067,641, which are herein incorporated by reference in their entireties). In some embodiments, suitable antisense oligonucleotides are 2′-O-methoxyethyl “gapmers” which contain 2′-O-methoxyethyl-modified ribonucleotides on both 5′ and 3′ ends with at least ten deoxyribonucleotides in the center. These “gapmers” are capable of triggering RNase H-dependent degradation mechanisms of RNA targets. Other modifications of antisense oligonucleotides to enhance stability and improve efficacy, such as those described in U.S. Pat. No. 6,838,283, which is herein incorporated by reference in its entirety, are known in the art and are suitable for use in the methods of the invention. Antisense oligonucleotides may comprise a sequence that is at least partially complementary to a PTGES3 target sequence, e.g., at least about 75%, 80%, 85%, 90%, 91%, 92%, 93%, 94%, 95%, 96%, 97%, 98%, or 99% complementary to the PTGES3 target sequence. In some embodiments, the antisense oligonucleotide may be substantially complementary to the PTGES3 target sequence, that is at least about 95%, 96%, 97%, 98%, or 99% complementary to a target polynucleotide sequence. In one embodiment, the antisense oligonucleotide comprises a sequence that is 100% complementary to the PTGES3 target sequence.

In another embodiment, the inhibitor of PTGES3 is an inhibitory RNA molecule (e.g., a miRNA, a siRNA, a piRNA, or a snRNA) having a single-stranded or double-stranded region that is at least partially complementary to the target sequence of PTGES3, e.g., about 75%, 80%, 85%, 90%, 91%, 92%, 93%, 94%, 95%, 96%, 97%, 98%, or 99% complementary to the target sequence of PTGES3. In some embodiments, the inhibitory RNA comprises a sequence that is substantially complementary to the target sequence of PTGES3, e.g., about 95%, 96%, 97%, 98%, or 99% complementary to a target polynucleotide sequence. In other embodiments, the inhibitory RNA molecule may contain a region that has 100% complementarity to the target sequence. The inhibitory molecules may target the PTGES3 sequence of SEQ ID NO:1. In certain embodiments, the inhibitory RNA molecule may be a double-stranded, small interfering RNA or a short hairpin RNA molecule (shRNA) comprising a stem-loop structure.

Inhibitors can be detectably labeled by well-known techniques. Detectable labels include, for example, radioactive isotopes, fluorescent labels, chemiluminescent labels, bioluminescent labels and enzyme labels. Such labeled inhibitors can be used to determine cellular uptake efficiency, quantitate binding of inhibitors at target sites, or visualize inhibitor localization.

### CRISPR System for Targeting PTGES3 in Prostate Cancer Cells

In some embodiments, a CRISPR/Cas system is used to inactivate or reduce expression of an endogenous PTGES3 gene in a prostate cancer cell. For example, a CRISPR/Cas system can be used to delete, inactivate, or mutate an endogenous PTGES3 gene to eliminate or reduce PTGES3 gene expression or protein activity. Genome modification can be performed, for example, using homology directed repair (HDR) with a donor polynucleotide comprising a sequence comprising an intended genome edit flanked by a pair of homology arms responsible for targeting the donor polynucleotide to the target locus (e.g., PTGES3 gene) to be edited in a prostate cancer cell. The donor polynucleotide typically comprises a 5′ homology arm that hybridizes to a 5′ genomic target sequence and a 3′ homology arm that hybridizes to a 3′ genomic target sequence. The homology arms are referred to herein as 5′ and 3′ (i.e., upstream and downstream) homology arms, which relates to the relative position of the homology arms to the nucleotide sequence comprising the intended edit within the donor polynucleotide. The 5′ and 3′ homology arms hybridize to regions within the target locus in the genomic DNA to be modified, which are referred to herein as the “5′ target sequence” and “3′ target sequence,” respectively.

The homology arm must be sufficiently complementary for hybridization to the target sequence to mediate homologous recombination between the donor polynucleotide and genomic DNA at the target locus. For example, a homology arm may comprise a nucleotide sequence having at least about 80-100% sequence identity to the corresponding genomic target sequence, including any percent identity within this range, such as at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity thereto, wherein the nucleotide sequence comprising the intended edit is integrated into the genomic DNA by HDR at the genomic target locus recognized (i.e., sufficiently complementary for hybridization) by the 5′ and 3′ homology arms.

In certain embodiments, the corresponding homologous nucleotide sequences in the genomic target sequence (i.e., the “5′ target sequence” and “3′ target sequence”) flank a specific site for cleavage and/or a specific site for introducing the intended edit. The distance between the specific cleavage site and the homologous nucleotide sequences (e.g., each homology arm) can be several hundred nucleotides. In some embodiments, the distance between a homology arm and the cleavage site is 200 nucleotides or less (e.g., 0, 10, 20, 30, 50, 75, 100, 125, 150, 175, and 200 nucleotides). In most cases, a smaller distance may give rise to a higher gene targeting rate. In a preferred embodiment, the donor polynucleotide is substantially identical to the target genomic sequence, across its entire length except for the sequence changes to be introduced to a portion of the genome that encompasses both the specific cleavage site and the portions of the genomic target sequence to be altered.

A homology arm can be of any length, e.g. 10 nucleotides or more, 50 nucleotides or more, 100 nucleotides or more, 250 nucleotides or more, 300 nucleotides or more, 350 nucleotides or more, 400 nucleotides or more, 450 nucleotides or more, 500 nucleotides or more, 1000 nucleotides (1 kb) or more, 5000 nucleotides (5 kb) or more, 10000 nucleotides (10 kb) or more, etc. In some instances, the 5′ and 3′ homology arms are substantially equal in length to one another, e.g. one may be 30% shorter or less than the other homology arm, 20% shorter or less than the other homology arm, 10% shorter or less than the other homology arm, 5% shorter or less than the other homology arm, 2% shorter or less than the other homology arm, or only a few nucleotides less than the other homology arm. In other instances, the 5′ and 3′ homology arms are substantially different in length from one another, e.g. one may be 40% shorter or more, 50% shorter or more, sometimes 60% shorter or more, 70% shorter or more, 80% shorter or more, 90% shorter or more, or 95% shorter or more than the other homology arm.

The donor polynucleotide is used in combination with an RNA-guided nuclease, which is targeted to a particular genomic sequence (i.e., genomic target sequence to be modified) by a guide RNA (gRNA). A target-specific guide RNA comprises a nucleotide sequence that is complementary to a genomic target sequence, and thereby mediates binding of the nuclease-gRNA complex by hybridization at the target site. For example, the gRNA can be designed with a sequence complementary to a target sequence in the PTGES3 gene. In some embodiments, the gRNA is designed to with a sequence complementary to a cancer-specific PTGES3 mutation to target the nuclease-gRNA complex to the site of a mutation in a prostate cancer cell. The mutation may comprise an insertion, a deletion, or a substitution. For example, the mutation may include a single nucleotide variation, gene fusion, translocation, inversion, duplication, frameshift, missense, nonsense, or other mutation. The targeted minor allele may be a common genetic variant or a rare genetic variant. In certain embodiments, the gRNA is designed to selectively bind to a minor allele with single base-pair discrimination, for example, to allow binding of the nuclease-gRNA complex to a single nucleotide polymorphism (SNP). In particular, the gRNA may be designed to target disease-relevant mutations of interest for the purpose of genome editing to delete or deactivate the PTGES3 gene in a prostate cancer cell.

In certain embodiments, the RNA-guided nuclease used for genome modification is a CRISPR system Cas nuclease. Any RNA-guided Cas nuclease capable of catalyzing site-directed cleavage of DNA to allow integration of donor polynucleotides by the HDR mechanism can be used in genome editing, including CRISPR system type I, type II, or type III Cas nucleases. Examples of Cas proteins include Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, Cas12a (Cpf1), Cas12b (C2c1), Cas12c (C2c3), Cas12d (CasY), Cas12e (CasX), CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof.

In certain embodiments, a type II CRISPR system Cas9 endonuclease is used. Cas9 nucleases from any species, or biologically active fragments, variants, analogs, or derivatives thereof that retain Cas9 endonuclease activity (i.e., catalyze site-directed cleavage of DNA to generate double-strand breaks) may be used to perform genome modification as described herein. The Cas9 need not be physically derived from an organism, but may be synthetically or recombinantly produced. Cas9 sequences from a number of bacterial species are well known in the art and listed in the National Center for Biotechnology Information (NCBI) database. See, for example, NCBI entries for Cas9 from: Streptococcus pyogenes (WP_002989955, WP_038434062, WP_011528583); Campylobacter jejuni (WP_022552435, YP_002344900), Campylobacter coli (WP_060786116); Campylobacter fetus (WP_059434633); Corynebacterium ulcerans (NC_015683, NC_017317); Corynebacterium diphtheria (NC_016782, NC_016786); Enterococcus faecalis (WP_033919308); Spiroplasma syrphidicola (NC_021284); Prevotella intermedia (NC_017861); Spiroplasma taiwanense (NC_021846); Streptococcus iniae (NC_021314); Belliella baltica (NC_018010); Psychroflexus torquisl (NC_018721); Streptococcus thermophilus (YP_820832), Streptococcus mutans (WP_061046374, WP_024786433); Listeria innocua (NP_472073); Listeria monocytogenes (WP_061665472); Legionella pneumophila (WP_062726656); Staphylococcus aureus (WP_001573634); Francisella tularensis (WP_032729892, WP_014548420), Enterococcus faecalis (WP_033919308); Lactobacillus rhamnosus (WP_048482595, WP_032965177); and Neisseria meningitidis (WP_061704949, YP_002342100); all of which sequences (as entered by the date of filing of this application) are herein incorporated by reference. Any of these sequences or a variant thereof comprising a sequence having at least about 70-100% sequence identity thereto, including any percent identity within this range, such as 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% sequence identity thereto, can be used for genome editing, as described herein. See also Fonfara et al. (2014) Nucleic Acids Res. 42(4):2577-90; Kapitonov et al. (2015) J. Bacteriol. 198(5):797-807, Shmakov et al. (2015) Mol. Cell. 60(3):385-397, and Chylinski et al. (2014) Nucleic Acids Res. 42(10):6091-6105); for sequence comparisons and a discussion of genetic diversity and phylogenetic analysis of Cas9.

The CRISPR-Cas system naturally occurs in bacteria and archaea where it plays a role in RNA-mediated adaptive immunity against foreign DNA. The bacterial type II CRISPR system uses the endonuclease, Cas9, which forms a complex with a guide RNA (gRNA) that specifically hybridizes to a complementary genomic target sequence, where the Cas9 endonuclease catalyzes cleavage to produce a double-stranded break. Targeting of Cas9 typically further relies on the presence of a 5′ protospacer-adjacent motif (PAM) in the DNA at or near the gRNA-binding site.

The genomic target site will typically comprise a nucleotide sequence that is complementary to the gRNA, and may further comprise a protospacer adjacent motif (PAM). In certain embodiments, the target site comprises 20-30 base pairs in addition to a 3 base pair PAM. Typically, the first nucleotide of a PAM can be any nucleotide, while the two other nucleotides will depend on the specific Cas9 protein that is chosen. Exemplary PAM sequences are known to those of skill in the art and include, without limitation, NNG, NGN, NAG, and NGG, wherein N represents any nucleotide. In certain embodiments, the allele targeted by a gRNA comprises a mutation that creates a PAM within the allele, wherein the PAM promotes binding of the Cas9-gRNA complex to the allele.

In certain embodiments, the gRNA is 5-50 nucleotides, 10-30 nucleotides, 15-25 nucleotides, 18-22 nucleotides, or 19-21 nucleotides in length, or any length between the stated ranges, including, for example, 10, 11, 12, 13, 14, 15, 16, 17, 18, 19, 20, 21, 22, 23, 24, 25, 26, 27, 28, 29, 30, 31, 32, 33, 34, or 35 nucleotides in length. The guide RNA may be a single guide RNA comprising crRNA and tracrRNA sequences in a single RNA molecule, or the guide RNA may comprise two RNA molecules with crRNA and tracrRNA sequences residing in separate RNA molecules.

In another embodiment, the CRISPR nuclease from Prevotella and Francisella 1 (Cpf1, also known as Cas12a) is used. Cpf1 is another class II CRISPR/Cas system RNA-guided nuclease with similarities to Cas9 and may be used analogously. Unlike Cas9, Cpf1 does not require a tracrRNA and only depends on a crRNA in its guide RNA, which provides the advantage that shorter guide RNAs can be used with Cpf1 for targeting than Cas9. Cpf1 is capable of cleaving either DNA or RNA. The PAM sites recognized by Cpf1 have the sequences 5′-YTN-3′ (where “Y” is a pyrimidine and “N” is any nucleobase) or 5′-TTN-3′, in contrast to the G-rich PAM site recognized by Cas9. Cpf1 cleavage of DNA produces double-stranded breaks with a sticky-ends having a 4 or 5 nucleotide overhang. For a discussion of Cpf1, see, e.g., Ledford et al. (2015) Nature. 526 (7571):17-17, Zetsche et al. (2015) Cell. 163 (3):759-771, Murovec et al. (2017) Plant Biotechnol. J. 15(8):917-926, Zhang et al. (2017) Front. Plant Sci. 8:177, Fernandes et al. (2016) Postepy Biochem. 62(3):315-326; herein incorporated by reference.

Cas12b (C2c1) is another class II CRISPR/Cas system RNA-guided nuclease that may be used. C2c1, similarly to Cas9, depends on both a crRNA and tracrRNA for guidance to target sites. For a description of Cas12b, see, e.g., Shmakov et al. (2015) Mol Cell. 60(3):385-397, Zhang et al. (2017) Front Plant Sci. 8:177; herein incorporated by reference.

In yet another embodiment, an engineered RNA-guided Fokl nuclease may be used. RNA-guided Fokl nucleases comprise fusions of inactive Cas9 (dCas9) and the Fokl endonuclease (Fokl-dCas9), wherein the dCas9 portion confers guide RNA-dependent targeting on Fokl. For a description of engineered RNA-guided Fokl nucleases, see, e.g., Havlicek et al. (2017) Mol. Ther. 25(2):342-355, Pan et al. (2016) Sci Rep. 6:35794, Tsai et al. (2014) Nat Biotechnol. 32(6):569-576; herein incorporated by reference.

An RNA-guided nuclease can be provided in the form of a protein, such as the nuclease complexed with a gRNA, or provided by a nucleic acid encoding the RNA-guided nuclease, such as an RNA (e.g., messenger RNA) or DNA (expression vector). In some embodiments, the RNA-guided nuclease and the gRNA are both provided by vectors. Both can be expressed by a single vector or separately on different vectors. The vector(s) encoding the RNA-guided nuclease an gRNA may be included in a CRISPR expression system to target the PTGES3 gene in prostate cancer cells.

Codon usage may be optimized to improve production of an RNA-guided nuclease in a particular cell or organism. For example, a nucleic acid encoding an RNA-guided nuclease or reverse transcriptase can be modified to substitute codons having a higher frequency of usage in a human cell, a non-human cell, a mammalian cell, a rodent cell, a mouse cell, a rat cell, or any other host cell of interest, as compared to the naturally occurring polynucleotide sequence. When a nucleic acid encoding the RNA-guided nuclease is introduced into cells (e.g., prostate cancer cells), the protein can be transiently, conditionally, or constitutively expressed in the cell.

In another embodiment, CRISPR interference (CRISPRi) is used to repress PTGES3 gene expression. CRISPRi is performed with a complex of a catalytically inactive Cas9 (dCas9) with a guide RNA that targets the PTGES3 gene. An engineered nuclease-deactivated Cas9 (dCas9) is used to allow sequence-specific targeting without cleavage. Nuclease-deactivated forms of Cas9 may be engineered by mutating catalytic residues at the active site of Cas9 to destroy nuclease activity. Any such nuclease deficient Cas9 protein from any species may be used as long as the engineered dCas9 retains gRNA-mediated sequence-specific targeting. In particular, the nuclease activity of Cas9 from Streptococcus pyogenes can be deactivated by introducing two mutations (D10A and H841A) in the RuvC1 and HNH nuclease domains. Other engineered dCas9 proteins may be produced by similarly mutating the corresponding residues in other bacterial Cas9 isoforms. Fora description of engineered nuclease-deactivated forms of Cas9, see, e.g., Qi et al. (2013) Cell 152:1173-1183, Dominguez et al. (2016) Nat. Rev. Mol. Cell. Biol. 17(1):5-15; herein incorporated by reference in their entireties.

The dCas9 protein can be designed to target the PTGES3 gene by altering its guide RNA sequence. A target-specific single guide RNA (sgRNA) comprises a nucleotide sequence that is complementary to a target site, and thereby mediates binding of the dCas9-sgRNA complex by hybridization at the target site. CRISPRi can be used to sterically repress transcription by blocking either transcriptional initiation or elongation by designing a sgRNA with a sequence complementary to a PTGES3 promoter or exonic sequence. The sgRNA may be complementary to the non-template strand or the template strand, but preferably is complementary to the non-template strand to more strongly repress transcription.

The target site will typically comprise a nucleotide sequence that is complementary to the sgRNA, and may further comprise a protospacer adjacent motif (PAM). In certain embodiments, the target site comprises 20-30 base pairs in addition to a 3 base pair PAM. Typically, the first nucleotide of a PAM can be any nucleotide, while the two other nucleotides will depend on the specific Cas9 protein that is chosen. Exemplary PAM sequences are known to those of skill in the art and include, without limitation, NNG, NGN, NAG, and NGG, wherein N represents any nucleotide.

In certain embodiments, the sgRNA comprises 5-50 nucleotides, 10-30 nucleotides, 15-25 nucleotides, 18-22 nucleotides, 19-21 nucleotides, and any length between the stated ranges, including, for example, 10, 11, 12, 13, 14, 15, 16, 17, 18, 19, 20, 21, 22, 23, 24, 25, 26, 27, 28, 29, or 30 nucleotides.

The sgRNAs are readily synthesized by standard techniques, e.g., solid phase synthesis via phosphoramidite chemistry, as disclosed in U.S. Pat. Nos. 4,458,066 and 4,415,732, incorporated herein by reference; Beaucage et al., Tetrahedron (1992) 48:2223-2311; and Applied Biosystems User Bulletin No. 13 (1 Apr. 1987). Other chemical synthesis methods include, for example, the phosphotriester method described by Narang et al., Meth. Enzymol. (1979) 68:90 and the phosphodiester method disclosed by Brown et al., Meth. Enzymol. (1979) 68:109.

In some embodiments, the dCas9 is fused to a transcriptional repressor domain capable of further repressing transcription of the PTGES3 gene, e.g., by inducing heterochromatinization. For example, a Kruppel associated box (KRAB) can be fused to dCas9 to repress transcription of a target gene in human cells (see, e.g., Gilbert et al. (2013) Cell. 154 (2): 442-45, O'Geen et al. (2017) Nucleic Acids Res. 45(17):9901-9916; herein incorporated by reference).

Alternatively, dCas9 can be used to introduce epigenetic changes that reduce PTGES3 gene expression by fusion of dCas9 to an epigenetic modifier such as a chromatin-modifying epigenetic enzyme. The promoter for PTGES3 can be silenced, for example, by methylation or acetylation (e.g. histone H3 lysine 9 [H3K9] methylation, histone H3 lysine 27 [H3K27] methylation, and/or DNA methylation). For example, fusion of dCas9 to a DNA methyltransferase such as DNA methyltransferase 3 alpha (DNMT3A) or a chimeric Dnmt3a/Dnmt3L methyltransferase (DNMT3A3L) allows targeted DNA methylation. Fusion of dCas9 to histone demethylase LSD1 allows targeted histone demethylation (see, e.g., Liu et al. (2016) Cell 167(1):233-247, Lo et al. (2017) F1000Res. 6. pii: F1000 Faculty Rev-747, and Stepper et al. (2017) Nucleic Acids Res. 45(4):1703-1713; herein incorporated by reference).

In yet other embodiments, an RNA-targeting CRISPR-Cas13 system is used to perform RNA interference to reduce PTGES3 expression. Members of the Cas13 family are RNA-guided RNases containing two HEPN domains having RNase activity. In particular, Cas13a (C2c2), Cas13b (C2c6), and Cas13d can be used for RNA knockdown. Cas13 proteins can be made to target and cleave PTGES3 transcribed RNA using a gRNA with complementarity to the target transcript sequence. The gRNA is typically about 64 nucleotides in length with a short hairpin crRNA and a 28-30 nucleotide spacer that is complementary to the target site on the PTGES3 RNA transcript. Cas13 recognition and cleavage of a target transcript results in degradation of the transcript as well as nonspecific degradation of any nearby transcripts. See, e.g., Abudayyeh et al. (2017) Nature 550:280-284, Hameed et al. (2019) Microb. Pathog. 133:103551, Wang et al. (2019) Biotechnol Adv. 37(5):708-729, Aman et al. (2018) Viruses 10(12). pii: E732, and Zhang et al. (2018) Cell 175(1):212-223; herein incorporated by reference.

### Determining Levels of Expression of PTGES3

An “effective amount” of a PTGES3 inhibitory nucleic acid (e.g., microRNA, siRNA, piRNA, snRNA, antisense oligonucleotide) or a CRISPR system targeting the PTGES3 gene (e.g., Cas9, Cas12a), RNA (e.g., Cas13), or epigenome (e.g., dCas9 fusion protein) is an amount sufficient to effect beneficial or desired results, such as an amount that reduces PTGES3 activity, for example, by interfering with transcription of PTGES3 or interfering with translation of PTGES3. In some embodiments, a PTGES3 inhibitory nucleic acid or CRISPR system reduces the PTGES3 mRNA levels or protein levels by at least about 10% to about 100%, 20% to about 100%, 30% to about 100%, 40% to about 100%, 50% to about 100%, 60% to about 100%, 70% to about 100%, 10% to about 90%, 20% to about 85%, 40% to about 84%, 60% to about 90%, including any percent within these ranges, such as but not limited to 15%, 20%, 25%, 30%, 35%, 40%, 45%, 50%, 55%, 60%, 65%, 70%, 75%, 80%, 85%, 90%, 95%, 96%, 97%, 98%, and 99%.

Any convenient protocol may be used for evaluating PTGES3 expression by detecting PTGES3 protein or mRNA levels in the presence or absence of a candidate PTGES3 inhibitor. For measuring protein levels in a sample, various antibody-based methods, including without limitation immunoassays, e.g., enzyme-linked immunosorbent assays (ELISAs), immunohistochemistry, and flow cytometry (FACS) may be used. Any convenient antibody can be used that specifically binds to the PTGES3 protein. The terms “specifically binds” or “specific binding” as used herein refer to preferential binding to a molecule relative to other molecules or moieties in a solution or reaction mixture (e.g., an antibody specifically binds to a particular polypeptide or epitope relative to other available polypeptides or epitopes). In some embodiments, the affinity of one molecule for another molecule to which it specifically binds is characterized by a K_{d} (dissociation constant) of 10⁻⁵ M or less (e.g., 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, or 10⁻¹⁶ M or less). By “Affinity” it is meant the strength of binding, increased binding affinity being correlated with a lower K_{d}.

While a variety of different manners of assaying for protein levels are known in the art, one representative and convenient type of protocol for assaying protein levels is the enzyme-linked immunosorbent assay (ELISA). In ELISA and ELISA-based assays, one or more antibodies specific for the proteins of interest may be immobilized onto a selected solid surface, preferably a surface exhibiting a protein affinity such as the wells of a polystyrene microtiter plate. After washing to remove incompletely adsorbed material, the assay plate wells are coated with a non-specific “blocking” protein that is known to be antigenically neutral with regard to the test sample such as bovine serum albumin (BSA), casein or solutions of powdered milk. This allows for blocking of non-specific adsorption sites on the immobilizing surface, thereby reducing the background caused by non-specific binding of antigen onto the surface. After washing to remove unbound blocking protein, the immobilizing surface is contacted with the sample to be tested under conditions that are conducive to immune complex (antigen/antibody) formation. Such conditions include diluting the sample with diluents such as BSA or bovine gamma globulin (BGG) in phosphate buffered saline (PBS)/Tween or PBS/Triton-X 100, which also tend to assist in the reduction of nonspecific background, and allowing the sample to incubate for about 2-4 hours at temperatures on the order of about 25°-27° C. (although other temperatures may be used). Following incubation, the antisera-contacted surface is washed so as to remove non-immunocomplexed material. An exemplary washing procedure includes washing with a solution such as PBS/Tween, PBS/Triton-X 100, or borate buffer. The occurrence and amount of immunocomplex formation may then be determined by subjecting the bound immunocomplexes to a second antibody having specificity for the target that differs from the first antibody and detecting binding of the second antibody. In certain embodiments, the second antibody will have an associated enzyme, e.g. urease, peroxidase, or alkaline phosphatase, which will generate a color precipitate upon incubating with an appropriate chromogenic substrate. For example, a urease or peroxidase-conjugated anti-human IgG may be employed, for a period of time and under conditions which favor the development of immunocomplex formation (e.g., incubation for 2 hours at room temperature in a PBS-containing solution such as PBS/Tween). After such incubation with the second antibody and washing to remove unbound material, the amount of label is quantified, for example by incubation with a chromogenic substrate such as urea and bromocresol purple in the case of a urease label or 2,2′-azino-di-(3-ethyl-benzthiazoline)-6-sulfonic acid (ABTS) and H₂O₂, in the case of a peroxidase label. Quantitation is then achieved by measuring the degree of color generation, e.g., using a visible spectrum spectrophotometer. The preceding format may be altered by first binding the sample to the assay plate. Then, primary antibody is incubated with the assay plate, followed by detecting of bound primary antibody using a labeled second antibody with specificity for the primary antibody.

The solid substrate upon which the antibody or antibodies are immobilized can be made of a wide variety of materials and in a wide variety of shapes, e.g., microtiter plate, microbead, dipstick, resin particle, etc. The substrate may be chosen to maximize signal to noise ratios, to minimize background binding, as well as for ease of separation and cost. Washes may be effected in a manner most appropriate for the substrate being used, for example, by removing a bead or dipstick from a reservoir, emptying or diluting a reservoir such as a microtiter plate well, or rinsing a bead, particle, chromatographic column or filter with a wash solution or solvent.

Alternatively, non-ELISA based-methods for measuring the levels of the PTGES3 protein in a sample may be employed, and any convenient method may be used. Representative examples known to one of ordinary skill in the art include but are not limited to other immunoassay techniques such as radioimmunoassays (RIA), sandwich immunoassays, fluorescent immunoassays, enzyme multiplied immunoassay technique (EMIT), capillary electrophoresis immunoassays (CEIA), and immunoprecipitation assays; mass spectrometry, or tandem mass spectrometry, proteomic arrays, xMAP microsphere technology, western blotting, immunohistochemistry, flow cytometry, cytometry by time-of-flight (CyTOF), multiplexed ion beam imaging (MIBI), and detection in body fluid by electrochemical sensor. In, for example, flow cytometry methods, the quantitative level of gene products of the one or more genes of interest are detected on cells in a cell suspension by lasers. As with ELISAs and immunohistochemistry, antibodies (e.g., monoclonal antibodies) that specifically bind the polypeptides encoded by the genes of interest are used in such methods.

As another example, electrochemical sensors may be employed. In such methods, a capture aptamer or an antibody that is specific for a target protein (the “analyte”) is immobilized on an electrode. A second aptamer or antibody, also specific for the target protein, is labeled with, for example, pyrroquinoline quinone glucose dehydrogenase ((PQQ)GDH). The sample of body fluid is introduced to the sensor either by submerging the electrodes in body fluid or by adding the sample fluid to a sample chamber, and the analyte allowed to interact with the labeled aptamer/antibody and the immobilized capture aptamer/antibody. Glucose is then provided to the sample, and the electric current generated by (PQQ)GDH is observed, where the amount of electric current passing through the electrochemical cell is directly related to the amount of analyte captured at the electrode.

Flow cytometry can be used to distinguish subpopulations of cells expressing different cellular markers and to determine their frequency in a population of cells. Typically, whole cells are incubated with antibodies that specifically bind to the cellular markers. The antibodies can be labeled, for example, with a fluorophore, isotope, or quantum dot to facilitate detection of the cellular markers. The cells are then suspended in a stream of fluid and passed through an electronic detection apparatus. In addition, fluorescence-activated cell sorting (FACS) can be used to sort a heterogeneous mixture of cells into separate containers. (See, e.g., Shapiro Practical Flow Cytometry, Wiley-Liss, 4^{th} edition, 2003; Loken Immunofluorescence Techniques in Flow Cytometry and Sorting, Wiley, 2^{nd} edition,1990; Flow Cytometry: Principles and Applications, (ed. Macey), Humana Press 1^{st} edition, 2007; herein incorporated by reference in their entireties.)

In other embodiments, the amount or level in the sample of mRNA encoded by the PTGES3 gene is determined. Any convenient method for measuring mRNA levels in a sample may be used, e.g. hybridization-based methods, e.g. northern blotting and in situ hybridization (Parker & Barnes, Methods in Molecular Biology 106:247-283 (1999)), RNase protection assays (Hod, Biotechniques 13:852-854 (1992)), and PCR-based methods (e.g. reverse transcription PCR (RT-PCR) (Weis et al., Trends in Genetics 8:263-264 (1992)).

For measuring mRNA levels, the starting material may be total RNA, i.e. unfractionated RNA, or poly A+ RNA isolated from a suspension of cells (e.g. prostate cancer cells). General methods for mRNA extraction are well known in the art and are disclosed in standard textbooks of molecular biology, including Ausubel et al., Current Protocols of Molecular Biology, John Wiley and Sons (1997). RNA isolation can also be performed using a purification kit, buffer set and protease from commercial manufacturers, according to the manufacturer's instructions. For example, RNA from cell suspensions can be isolated using Qiagen RNeasy mini-columns, and RNA from cell suspensions or homogenized tissue samples can be isolated using the TRIzol reagent-based kits (Invitrogen), MasterPure Complete DNA and RNA Purification Kit (EPICENTRE, Madison, Wis.), Paraffin Block RNA Isolation Kit (Ambion, Inc.) or RNA Stat-60 kit (Tel-Test).

The mRNA levels may be measured by any convenient method. Examples of methods for measuring mRNA levels may be found in, e.g., the field of differential gene expression analysis. One representative and convenient type of protocol for measuring mRNA levels is array-based gene expression profiling. Such protocols are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively.

Specific hybridization technology which may be employed in the subject methods includes that described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the phenotype determinative genes whose expression is being assayed is contacted with target nucleic acids as described above. Contact is carried out under hybridization conditions, e.g., stringent hybridization conditions, and unbound nucleic acid is then removed. The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

The resultant pattern of hybridized nucleic acid provides information regarding expression for each of the genes that have been probed, where the expression information is in terms of whether or not the gene is expressed and, typically, at what level, where the expression data, i.e., expression profile (e.g., in the form of a transcriptosome), may be both qualitative and quantitative.

Additionally or alternatively, non-array based methods for quantitating the level of one or more nucleic acids in a sample may be employed. These include those based on amplification protocols, e.g., Polymerase Chain Reaction (PCR)-based assays, including quantitative PCR, reverse-transcription PCR (RT-PCR), real-time PCR, and the like, e.g. TaqMan, RT-PCR, MassARRAY System, BeadArray technology, and Luminex technology; and those that rely upon hybridization of probes to filters, e.g. Northern blotting and in situ hybridization. Serial Analysis Gene Expression (SAGE) can also be used to determine RNA abundances in a cell sample. See, e.g., Velculescu et al., 1995, Science 270:484-7; Carulli, et al., 1998, Journal of Cellular Biochemistry Supplements 30/31:286-96; herein incorporated by reference in their entireties. SAGE analysis does not require a special device for detection, can be used for simultaneously detecting the expression of large numbers of transcription products.

### Screening Inhibitors for their Effects on Prostate Cancer

In some embodiments, a test agent that inhibits PTGES3 activity is further tested for its ability to inhibit growth of prostate cancer cells in a cell-based assay. In these embodiments, a test agent of interest is contacted with the prostate cancer cells; and the effect, if any, of the test agent on the prostate cancer cells is determined.

For example, a population of prostate cancer cells can be cultured in vitro in the presence of an effective dose of an inhibitor of PTGES3 activity. The effect on growth, proliferation, and/or AR abundance may be assayed as described in the examples. The PTGES3 inhibitor is added to the culture medium, and the culture medium is maintained under conventional conditions suitable for growth of the prostate cancer cells. Various commercially available systems have been developed for the growth of mammalian cells to provide for removal of adverse metabolic products, replenishment of nutrients, and maintenance of oxygen. By employing these systems, the medium may be maintained as a continuous medium, so that the concentrations of the various ingredients are maintained relatively constant or within a prescribed range.

In some embodiments, a test compound identified as an inhibitor of PTGES3 in cell-based or cell-free assays is further tested for its efficacy in treating prostate cancer in vivo, e.g., in an animal such as an animal model for prostate cancer. For example, an agent that inhibits PTGES3 biological activity or expression, identified as described herein, can be used in an animal model to determine the efficacy, toxicity, or side effects of treatment with such an agent. Alternatively, an agent identified, as described herein, can be used in an animal model to determine the mechanism of action of such an agent. Monitoring the efficacy of agents (e.g., drugs) on prostate cancer can be applied not only in basic drug screening, but also in clinical trials. Furthermore, this disclosure pertains to uses of novel agents identified by the above-described screening assays for treatment of prostate cancer.

### Pharmaceutical Compositions

PTGES3 inhibitors, identified by the screening methods described herein, can be formulated into pharmaceutical compositions optionally comprising one or more pharmaceutically acceptable excipients. Exemplary excipients include, without limitation, carbohydrates, inorganic salts, antimicrobial agents, antioxidants, surfactants, buffers, acids, bases, and combinations thereof. Excipients suitable for injectable compositions include water, alcohols, polyols, glycerine, vegetable oils, phospholipids, and surfactants. A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate dibasic, and combinations thereof.

A composition of the invention can also include an antimicrobial agent for preventing or deterring microbial growth. Nonlimiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the composition as well. Antioxidants are used to prevent oxidation, thereby preventing the deterioration of the PTGES3 inhibitor, or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant can be present as an excipient. Exemplary surfactants include: polysorbates, such as “Tween 20” and “Tween 80,” and pluronics such as F68 and F88 (BASF, Mount Olive, New Jersey); sorbitan esters; lipids, such as phospholipids such as lecithin and other phosphatidylcholines, phosphatidylethanolamines (although preferably not in liposomal form), fatty acids and fatty esters; steroids, such as cholesterol; chelating agents, such as EDTA; and zinc and other such suitable cations.

Acids or bases can be present as an excipient in the composition. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of the PTGES3 inhibitor (e.g., when contained in a drug delivery system) in the composition will vary depending on a number of factors but will optimally be a therapeutically effective dose when the composition is in a unit dosage form or container (e.g., a vial). A therapeutically effective dose can be determined experimentally by repeated administration of increasing amounts of the composition in order to determine which amount produces a clinically desired endpoint.

The amount of any individual excipient in the composition will vary depending on the nature and function of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects. Generally, however, the excipient(s) will be present in the composition in an amount of about 1% to about 99% by weight, preferably from about 5% to about 98% by weight, more preferably from about 15 to about 95% by weight of the excipient, with concentrations less than 30% by weight most preferred. These foregoing pharmaceutical excipients along with other excipients are described in “Remington: The Science & Practice of Pharmacy”, 19th ed., Williams & Williams, (1995), the “Physician's Desk Reference”, 52nd ed., Medical Economics, Montvale, N.J. (1998), and Kibbe, A. H., Handbook of Pharmaceutical Excipients, 3rd Edition, American Pharmaceutical Association, Washington, D.C., 2000.

The compositions encompass all types of formulations and in particular those that are suited for injection, e.g., powders or lyophilates that can be reconstituted with a solvent prior to use, as well as ready for injection solutions or suspensions, dry insoluble compositions for combination with a vehicle prior to use, and emulsions and liquid concentrates for dilution prior to administration. Examples of suitable diluents for reconstituting solid compositions prior to injection include bacteriostatic water for injection, dextrose 5% in water, phosphate buffered saline, Ringer's solution, saline, sterile water, deionized water, and combinations thereof. With respect to liquid pharmaceutical compositions, solutions and suspensions are envisioned. Additional preferred compositions include those for oral, ocular, or localized delivery.

The pharmaceutical preparations herein can also be housed in a syringe, an implantation device, or the like, depending upon the intended mode of delivery and use. Preferably, the compositions comprising one or more PTGES3 inhibitors, or nucleic acids encoding them, are in unit dosage form, meaning an amount of a conjugate or composition of the invention appropriate for a single dose, in a premeasured or pre-packaged form.

The compositions herein may optionally include one or more additional agents, such as one or more other drugs for treating prostate cancer or other medications. For example, compounded preparations may include at least one PTGES3 inhibitor and one or more other drugs for treating prostate cancer such as antiandrogens including, without limitation, flutamide, nilutamide, bicalutamide, enzalutamide, apalutamide, and cyproterone acetate; medications that block the production of adrenal androgens including, without limitation, ketoconazole and aminoglutethimide; GnRH antagonists including, without limitation, abarelix and degarelixas; GnRH agonists including, without limitation, leuprorelin and goserelin; CYP17 inhibitors including, without limitation, abiraterone acetate; estrogens including, without limitation, diethylstilbestrol, fosfestrol, ethinylestradiol, ethinylestradiol sulfonate, polyestradiol phosphate, and estradiol undecylate; and other drugs for treating prostate cancer, or other medications used to treat a subject for a condition or disease. Alternatively, such agents can be contained in a separate composition from the composition comprising the PTGES3 inhibitor and co-administered concurrently, before, or after the composition comprising the PTGES3 inhibitor.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-47 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A method of screening for a prostaglandin E synthase 3 (PTGES3) inhibitor for treating prostate cancer, the method comprising:
a) contacting PTGES3 with a candidate agent; and
b) measuring inhibition of PTGES3 activity by the candidate agent.

2. The method of aspect 1, further comprising: contacting a population of prostate cancer cells with the candidate agent if the candidate agent inhibits the PTGES3 activity; and measuring proliferation, survival, or androgen receptor abundance in the population of prostate cancer cells, wherein reduced proliferation, survival, or androgen receptor abundance in presence of the candidate agent compared to that in a negative control population of prostate cancer cells that are not treated with the candidate agent indicates that the candidate agent has anti-cancer activity.

3. The method of aspect 1 or 2, wherein the candidate agent inhibits production of prostaglandin E2 from prostaglandin H2.

4. The method of any one of aspects 1 to 3, wherein the candidate agent inhibits PTGES3 protein chaperone activity.

5. The method of any one of aspects 1 to 4, wherein the candidate agent inhibits binding of the PTGES3 to an androgen receptor (AR) or modulation of AR activity by PTGES3.

6. The method of aspect 1 or 2, wherein the candidate agent inhibits PTGES3 gene expression.

7. The method of any one of aspects 1 to 6, wherein the candidate agent is a small molecule, a peptide, a protein, an aptamer, an antibody that specifically binds to PTGES3, an antibody mimetic, an inhibitory nucleic acid, or a Clustered Regularly Interspaced Short Palindromic Repeats (CRISPR) system.

8. The method of aspect 7, wherein the antibody is selected from the group consisting of a polyclonal antibody, a monoclonal antibody, a chimeric antibody, a humanized antibody, a F(ab) fragment, a F(ab′)2 fragment, a Fᵥ fragment, and a nanobody.

9. The method of aspect 7, wherein the inhibitory nucleic acid is selected from the group consisting of a small interfering RNA (siRNA), a microRNA (miRNA), a Piwi-interacting RNA (piRNA), a small nuclear RNA (snRNA), an antisense oligonucleotide, and a peptide nucleic acid.

10. The method of aspect 9, wherein the inhibitory nucleic acid inhibits PTGES3 transcription or protein translation.

11. The method of aspect 7, wherein the CRISPR system targets a PTGES3 gene or a PTGES3 RNA transcript, or makes epigenetic changes that reduce PTGES3 expression.

12. The method of aspect 11, wherein the CRISPR system comprises Cas9, Cas12a, Cas12d, Cas13a, Cas13b, Cas13d, or a dead Cas9 (dCas9).

13. A PTGES3 inhibitor identified by the method of any one of aspects 1 to 12.

14. A composition comprising the PTGES3 inhibitor of aspect 13 and a pharmaceutically acceptable excipient.

15. The composition of aspect 14, further comprising a pharmaceutically acceptable carrier selected from the group consisting of a cream, emulsion, gel, liposome, nanoparticle, or ointment.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention.

It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

### The Androgen Receptor: A Key Therapeutic Target for Aggressive Prostate Cancer

The androgen receptor (AR) is a central driver of prostate cancer^{1,2}. The majority of prostate cancers express AR throughout the course of the disease^{2,18-20,} and AR has been shown to promote transcriptional programs governing critical oncogenic phenotypes, such as proliferation, migration, and invasion²¹. Moreover, many key genes that drive prostate cancer (PCa), such as FOXA1 or HOXB13, promote tumor progression by regulating how AR binds to or activates its target genes. Collectively, these findings highlight the importance of AR biology in this disease.

Multiple phase III trials have demonstrated the benefit of AR-targeted therapies on patient survival³⁻¹². AR signaling inhibitors (ARSI) are now the standard of care for locally advanced⁶, recurrent⁷, non-metastatic castration-resistant^{3,11,12,} metastatic castration-sensitive⁸⁻¹⁰, and metastatic castration-resistant prostate cancer (mCRPC)^{4,5}. However, aggressive PCa invariably escape these therapies by reactivating AR signaling¹³. Recent analyses of clinical samples, from our group and others, have demonstrated that 84% of mCRPCs exhibit robust AR nuclear staining after resistance to abiraterone or enzalutamide, and 69% are AR-positive at autopsy^{22,23}. Therefore, identifying novel approaches to target AR is critical to improving outcomes for mCRPC patients.

We and other groups have also identified tumor alterations that reactivate AR signaling during treatment with an ARSI^{2,24-31}. These mechanisms of resistance include AR gene amplification ^{24,25,} AR enhancer amplification ^{2,26,27,} AR mutation²⁸, AR genomic structural rearrangements²⁹, AR splice variants³⁰, and polymorphisms in androgen metabolism genes³¹. All of these alterations invariably result in increased expression or increased activity of the AR protein. Thus, blocking AR protein expression represents a promising approach to overcoming these mechanisms of resistance in therapy-resistant mCRPC.

To investigate molecular determinants of AR protein expression, we utilized CRISPR gene editing approaches, previously pioneered by our team¹⁴, to develop the first reporter cell lines of endogenous AR protein expression. We then utilized two genome-wide CRISPRi screens, using this reporter system, to identify key regulators of AR protein expression. The top novel targetable hit from our screens was prostaglandin E synthase 3 (PTGES3).

### Example 2

### Prostate Cancer Cell Lines with an AR mNeonGreen2 Fluorescent Reporter

Accurate reporters of AR activity in prostate cancer cells are essential to developing the next generation of therapeutic approaches that target AR. Existing methods that fuse a large fluorescent reporter protein to an exogenously overexpressed target protein can affect target function, and are not ideal reporters^{33,34}. We previously developed an innovative split fluorescent protein tagging strategy enabling us to visualize and measure the expression of endogenous genes without antibodies or transgenes¹⁴. This approach has demonstrated many advantages, including significantly reduced perturbation to the genomic locus and the protein-protein interaction of the target compared to traditional methods¹⁴⁻¹⁶.

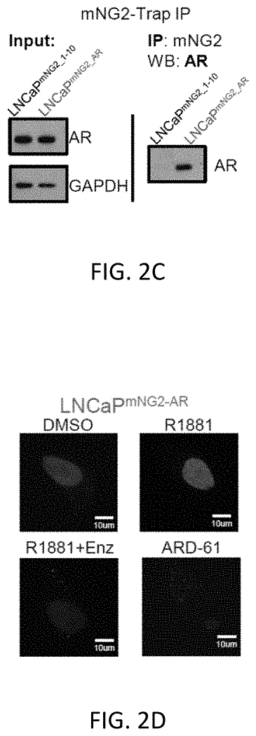

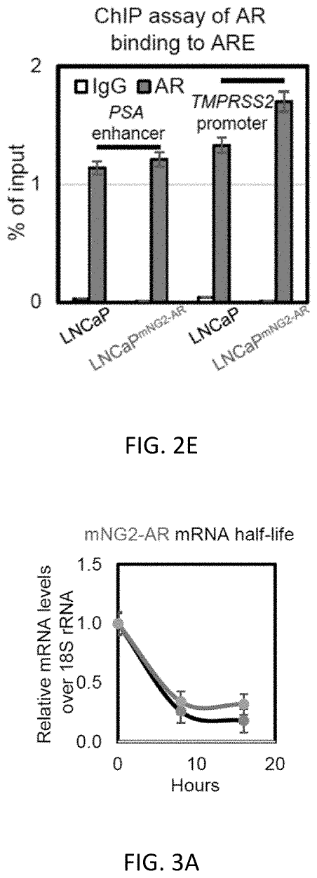

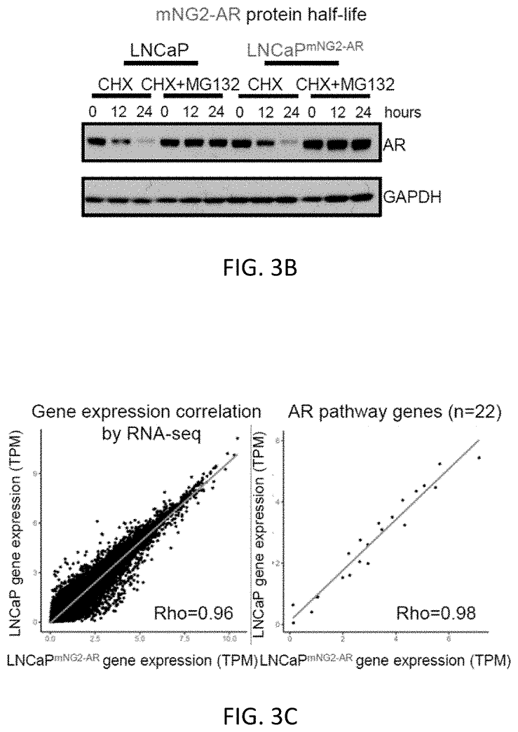

To study AR biology with fidelity, we have now established the first prostate cancer cell line models harboring an endogenous AR mNeonGreen2 (mNG2) fluorescent reporter enabling us to measure AR protein abundance and localization in live or fixed cells (FIGS. 2A-2B). We performed extensive genotypic and phenotypic characterization of this system in AR-tagged LNCaP^{mNG2-AR} _{and C}42B^{mNG2-AR} cells to demonstrate that the reporter is effective and does not perturb AR activity. The split mNG2 protein forms a protein complex with the tagged AR protein (FIG. 2C). We demonstrated that tagged AR protein biology responds as expected to AR stimulus by androgen (R1881) and AR repression by the antiandrogen enzalutamide (Enz) and the AR degrader ARD-61 (FIG. 2D). We confirmed that there were no significant differences in tagged AR mRNA levels, AR protein stability, or AR target gene expression by real-time PCR, western blots, and RNA-seq (FIG. 3). Lastly, we demonstrated that tagged AR function is unperturbed by using ChIP assays to show that AR and tagged AR bind indistinguishably to the PSA androgen response element and other known AR binding sites (FIG. 2E). To our knowledge, these models are the first to allow precise measurement of dynamic endogenous AR protein levels and localization, and are a valuable tool for the research community.

### Example 3

### Identification of Regulators of AR Expression using a Genome-Wide CRISPRi Screen

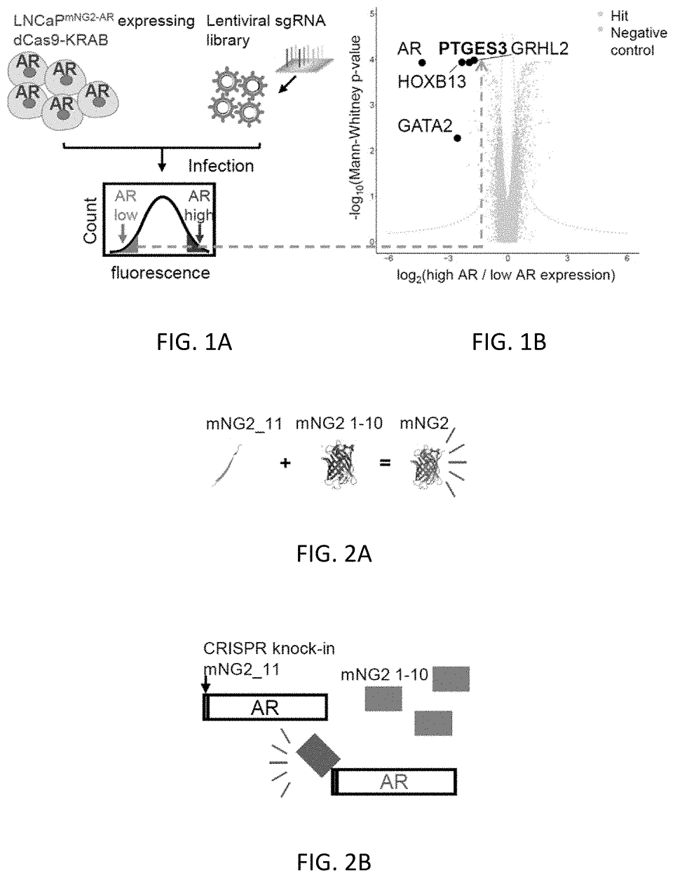

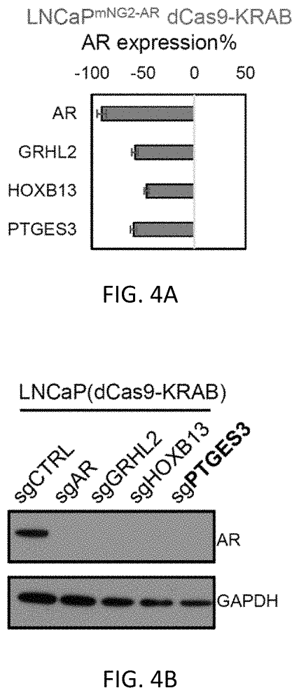

We next set out to use the LNCaP^{mNG2-AR} _{and C}42B^{mNG2-AR} cell lines to identify genes that regulate AR protein abundance. To this end, we stably expressed and optimized a dCas9-KRAB fusion construct in LNCaP^{mNG2-AR} and C42B^{mNG2-AR}. Control experiments demonstrated these cell lines have robust CRISPRi activity. We next performed genome-wide CRISPRi screens¹⁷, in LNcaP^{mNG2-AR} & C42B^{mNG2-AR} using fluorescence-activated cell sorting to identify genes whose down-regulation would modulate AR protein levels (FIG. 1A). As expected, AR and the upstream AR regulators HOXB13, GRHL2, and GATA2 were among the top hits (FIG. 1B), meaning that knockdown resulted in the greatest decrease in AR protein levels as measured by the reporter system. The emergence of these positive controls in an unbiased screen increased our confidence in high-scoring less-characterized hits, including prostaglandin E synthase 3 (PTGES3). We validated that repression of top gene hits with individual sgRNAs decreased AR protein levels (FIG. 4).

### Example 4

### PTGES3: A Top Regulator of AR Expression

We were intrigued by the identification of PTGES3 as a top hit. The mechanism by which PTGES3 stabilizes AR signaling is unknown. PTGES3 is reported in the literature to have multiple protein functions. The protein is reported to be a prostaglandin synthase enzyme which converts Prostaglandin H2 (PGH2) to Prostaglandin E2 (PGE2)³⁵; of note, other prostaglandin synthases in the same family as PTGES3 have been implicated in the production of androgens^{36,37}. PTGES3 also is reported to have HSP90-dependent and independent protein chaperone functions in stabilizing client proteins including hormone receptors³⁸⁻⁴⁰.

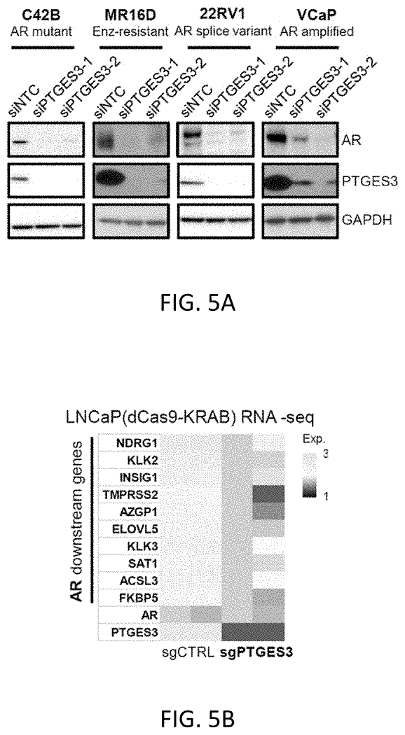

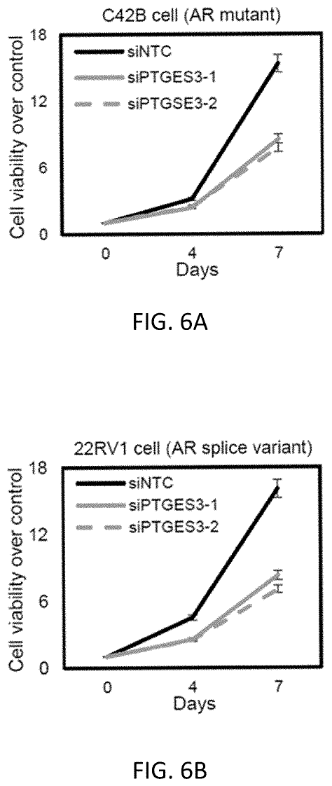

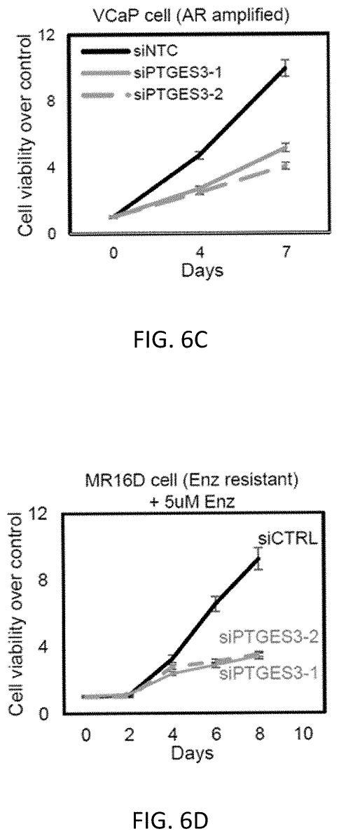

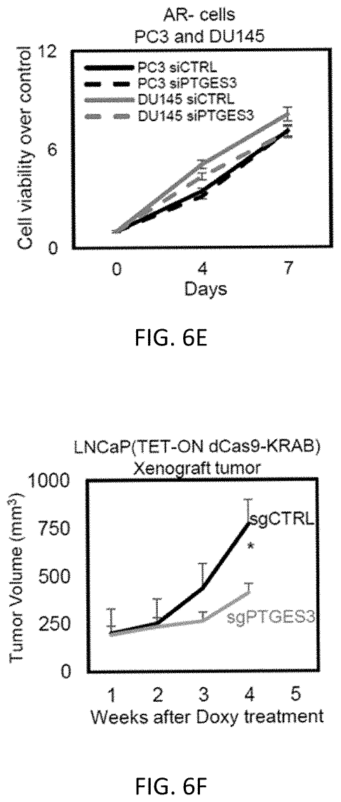

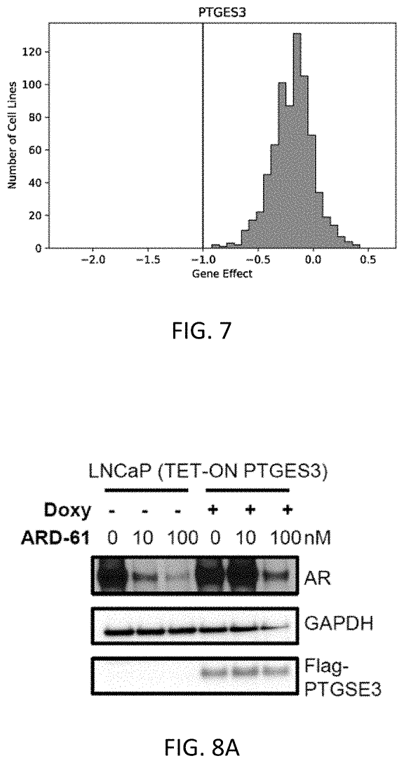

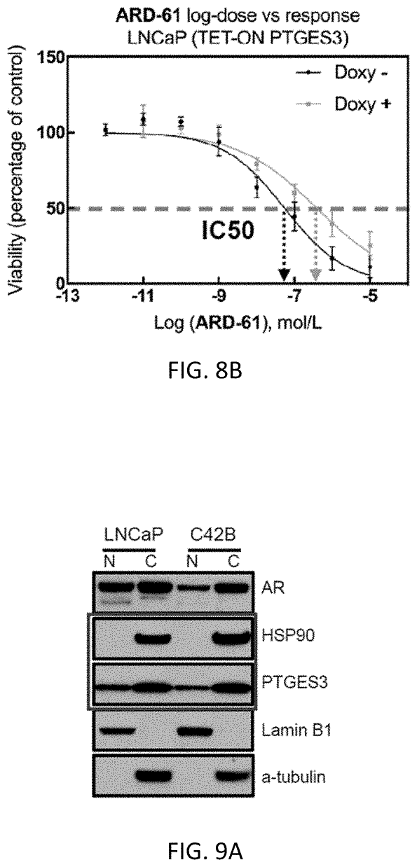

Preliminary experiments showed that repression of PTGES3 reduces AR protein levels but not AR mRNA levels, suggesting that PTGES3 regulates AR protein translation or stability (FIG. 5). This result is not unique to LNCaP and C42B cells, as PTGES3 knockdown also decreased AR protein levels in MR16D, 22RV1, and VCaP cells, all models of aggressive prostate cancer (FIG. 5A). Knockdown of PTGES3 decreases expression of AR target genes (FIG. 5B). Lastly as expected from the results above, PTGES3 is required for cell proliferation in vitro and in vivo in multiple AR-driven PCa models but not AR-independent PCa models such as PC3 cells (FIG. 6). These models represent diverse AR PCa biology as VCaP are AR amplified, 22RV1 express AR splice variants, LNCaP and C42B harbor AR mutations, and MR16D are resistant to the AR signaling inhibitor enzalutamide, suggesting a PTGES3 inhibitor could inhibit AR function across a wide range of mCRPC. Importantly, analysis of PTGES3 data from the Cancer Dependency map of genome-scale CRISPR knockout screens demonstrates that PTGES3 is not required for cell viability in the vast majority of non-prostate cell lines. This demonstrates that PTGES3 is conditionally essential in AR-driven prostate cancers and suggests PTGES3 inhibitors will be relatively non-toxic to most cell types (FIG. 7). Knockdown of PTGES3 sensitized cells to an AR-degrader suggesting that a PTGES3 inhibitor could be combined with AR inhibitors or degraders as a novel combination therapy for treatment of advanced AR-driven PCa or could be used as a monotherapy in the context of mCRPC (FIG. 8). These results demonstrate that PTGES3 is required for AR signaling, is conditionally essential in PCa and is a therapeutic target in AR-driven prostate cancer models.

### Example 5

### Mechanisms by which PTGES3 Regulates AR

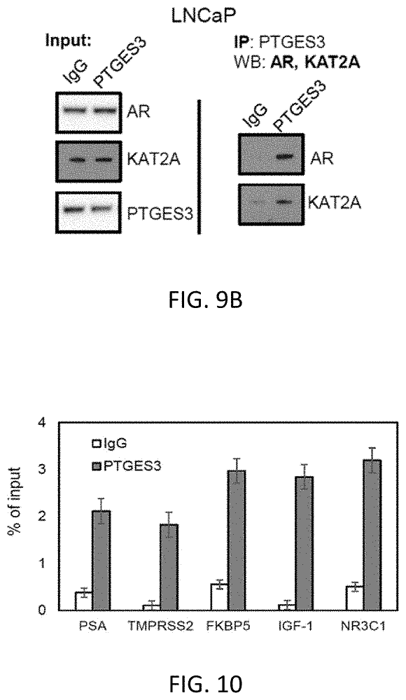

Previous studies in yeast and human cells have suggested that PTGES3 has an enzymatic function in prostaglandin synthesis as well as a HSP90 dependent and independent protein chaperone function. However, relatively little is known about PTGES3 in prostate cancer. It has been claimed that the PTGES3 enzymatic reaction occurs in the cytosol³⁵. However, IHC staining data from the Cell Atlas shows that in prostate cancers, PTGES3 is localized to the nucleus while the known AR-chaperone, HSP90, is cytosolic⁴¹. To measure PTGES3 localization we generated LNCaP and C42B nuclear and cytoplasmic extracts and then western blotted for AR, HSP90, PTGES3 as well as Lamin B1 (nuclear control) and tubulin (cytoplasmic control). Our results demonstrate that PTGES3 is localized in both the cytoplasm and nucleus (FIG. 9A). As expected, AR is localized in both the cytoplasm and nucleus while HSP90 is localized solely to the cytoplasm.

To investigate whether PTGES3 modulates AR directly or indirectly, we first tested whether PTGES3 interacts with AR. We immunoprecipitated (IP) PTGES3 from LNCaP cells and then western blotted for AR. Our result shows that PTGES3 and AR physically interact, although it remains to be determined whether this is a direct or indirect protein-protein interaction (FIG. 9B). Previous studies in yeast, rabbit reticulocyte lysates, and HEK293 cells have suggested that nuclear PTGES3 could bind to transcription factors as well as the chromatin opening factor KAT2A to facilitate transcription in a HSP90-independent manner^{35,42}. To investigate whether PTGES3 interacts with KAT2A in PCa, we also blotted our co-IP lysates for KAT2A. Our results demonstrate that PTGES3 interacts with KAT2A in LNCaP cells (FIG. 9B). This co-IP result suggests a PTGES3/AR/KAT2A protein complex is required for AR protein stability and function. PTGES3 is not predicted to bind directly to DNA; however, indirect protein/DNA interactions can be captured by dual cross-linked IP followed by real-time PCR for DNA sequences of interest⁴³. To begin to test the hypothesis that a PTGES3/AR/KAT2A protein complex is required for AR protein stability and function, we have performed a dual cross-linked IP for PTGES3 and then used real-time quantitative PCR to measure whether PTGES3 is enriched at known Androgen Response Elements (ARE). Our results demonstrate that PTGES3 is enriched at AREs that are hallmark AR targets (FIG. 10). Together these results suggest PTGES3 is required for AR stability and function in PCa, but the detailed mechanisms for this biology remain unclear.

### REFERENCES

1. Robinson, D. et al. Integrative clinical genomics of advanced prostate cancer. Cell 161, 1215-1228 (2015).

2. Quigley, D. A. et al. Genomic Hallmarks and Structural Variation in Metastatic Prostate Cancer. Cell 174, 758-769.e9 (2018).

3. Smith, M. R. et al. Apalutamide Treatment and Metastasis-free Survival in Prostate Cancer. N. Engl. J. Med. 378, 1408-1418 (2018).

4. Beer, T. M. et al. Enzalutamide in metastatic prostate cancer before chemotherapy. N. Engl. J. Med. 371, 424-433 (2014).

5. Ryan, C. J. et al. Abiraterone in metastatic prostate cancer without previous chemotherapy. N. Engl. J. Med. 368, 138-148 (2013).

6. Bolla, M. et al. Long-term results with immediate androgen suppression and external irradiation in patients with locally advanced prostate cancer (an EORTC study): a phase III randomised trial. Lancet 360, 103-106 (2002).

7. Shipley, W. U. et al. Radiation with or without Antiandrogen Therapy in Recurrent Prostate Cancer. N. Engl. J. Med. 376, 417-428 (2017).

8. Chi, K. N. et al. Apalutamide for Metastatic, Castration-Sensitive Prostate Cancer. N. Engl. J. Med. 381, 13-24 (2019).

9. Davis, I. D. et al. Enzalutamide with Standard First-Line Therapy in Metastatic Prostate Cancer. N. Engl. J. Med. 381, 121-131 (2019).

10. James, N. D. et al. Abiraterone for Prostate Cancer Not Previously Treated with Hormone Therapy. N. Engl. J. Med. 377, 338-351 (2017).

11. Sternberg, C. N. et al. Enzalutamide and Survival in Nonmetastatic, Castration-Resistant Prostate Cancer. N. Engl. J. Med. 382, 2197-2206 (2020).

12. Fizazi, K. et al. Darolutamide in Nonmetastatic, Castration-Resistant Prostate Cancer. N. Engl. J. Med. 380, 1235-1246 (2019).

13. Watson, P. A., Arora, V. K. & Sawyers, C. L. Emerging mechanisms of resistance to androgen receptor inhibitors in prostate cancer. Nat. Rev. Cancer 15, 701-711 (2015).

14. Kamiyama, D. et al. Versatile protein tagging in cells with split fluorescent protein. Nat Commun 7, 11046 (2016).

15. Feng, S. et al. Improved split fluorescent proteins for endogenous protein labeling. Nat Commun 8, 370 (2017).

16. Leonetti, M. D., Sekine, S., Kamiyama, D., Weissman, J. S. & Huang, B. A scalable strategy for high-throughput GFP tagging of endogenous human proteins. Proc. Natl. Acad. Sci. U.S.A. 113, E3501-3508 (2016).

17. Gilbert, L. A. et al. Genome-Scale CRISPR-Mediated Control of Gene Repression and Activation. Cell 159, 647-661 (2014).

18. Attard, G. et al. Characterization of ERG, AR and PTEN gene status in circulating tumor cells from patients with castration-resistant prostate cancer. Cancer Res. 69, 2912-2918 (2009).

19. Ruizeveld de Winter, J. A. et al. Androgen receptor status in localized and locally progressive hormone refractory human prostate cancer. Am. J. Pathol. 144, 735-746 (1994).

20. Sadi, M. V., Walsh, P. C. & Barrack, E. R. Immunohistochemical study of androgen receptors in metastatic prostate cancer. Comparison of receptor content and response to hormonal therapy. Cancer 67, 3057-3064 (1991).

21. Culig, Z. & Santer, F. R. Androgen receptor signaling in prostate cancer. Cancer Metastasis Rev. 33, 413-427 (2014).

22. Aggarwal, R. et al. Clinical and Genomic Characterization of Treatment-Emergent Small-Cell Neuroendocrine Prostate Cancer: A Multi-institutional Prospective Study. J. Clin. Oncol. 36, 2492-2503 (2018).

23. Bluemn, E. G. et al. Androgen Receptor Pathway-Independent Prostate Cancer Is Sustained through FGF Signaling. Cancer Cell 32, 474-489.e6 (2017).

24. Visakorpi, T. et al. In vivo amplification of the androgen receptor gene and progression of human prostate cancer. Nat. Genet. 9, 401-406 (1995).

25. Grasso, C. S. et al. The mutational landscape of lethal castration-resistant prostate cancer. Nature 487, 239-243 (2012).

26. Takeda, D. Y. et al. A Somatically Acquired Enhancer of the Androgen Receptor Is a Noncoding Driver in Advanced Prostate Cancer. Cell 174, 422-432.e13 (2018).

27. Viswanathan, S. R. et al. Structural Alterations Driving Castration-Resistant Prostate Cancer Revealed by Linked-Read Genome Sequencing. Cell 174, 433-447.e19 (2018).

28. Gregory, C. W., Johnson, R. T., Mohler, J. L., French, F. S. & Wilson, E. M. Androgen receptor stabilization in recurrent prostate cancer is associated with hypersensitivity to low androgen. Cancer Res. 61, 2892-2898 (2001).

29. Henzler, C. et al. Truncation and constitutive activation of the androgen receptor by diverse genomic rearrangements in prostate cancer. Nat Commun 7, 13668 (2016).

30. Dehm, S. M., Schmidt, L. J., Heemers, H. V., Vessella, R. L. & Tindall, D. J. Splicing of a novel androgen receptor exon generates a constitutively active androgen receptor that mediates prostate cancer therapy resistance. Cancer Res. 68, 5469-5477 (2008).

31. Chang, K.-H. et al. A gain-of-function mutation in DHT synthesis in castration-resistant prostate cancer. Cell 154, 1074-1084 (2013).

32. Attard, G. et al. Abiraterone Alone or in Combination With Enzalutamide in Metastatic Castration-Resistant Prostate Cancer With Rising Prostate-Specific Antigen During Enzalutamide Treatment. J. Clin. Oncol. 36, 2639-2646 (2018).

33. Cabantous, S., Terwilliger, T. C. & Waldo, G. S. Protein tagging and detection with engineered self-assembling fragments of green fluorescent protein. Nat. Biotechnol. 23, 102-107 (2005).

34. Waldo, G. S., Standish, B. M., Berendzen, J. & Terwilliger, T. C. Rapid protein-folding assay using green fluorescent protein. Nat. Biotechnol. 17, 691-695 (1999).

35. Tanioka, T., Nakatani, Y., Semmyo, N., Murakami, M. & Kudo, I. Molecular Identification of Cytosolic Prostaglandin E2 Synthase That Is Functionally Coupled with Cyclooxygenase-1 in Immediate Prostaglandin E2Biosynthesis. J. Biol. Chem. 275, 32775-32782 (2000).

36. Penning, T. M. AKR1C3 (type 5 173-hydroxysteroid dehydrogenase/prostaglandin F synthase): Roles in malignancy and endocrine disorders. Mol. Cell. Endocrinol. 489, 82-91 (2019).

37. Jeong, K. H., Jung, J. H., Kim, J. E. & Kang, H. Prostaglandin D2-Mediated DP2 and AKT Signal Regulate the Activation of Androgen Receptors in Human Dermal Papilla Cells. Int J Mol Sci 19, (2018).

38. Freeman, B. C. & Yamamoto, K. R. Disassembly of transcriptional regulatory complexes by molecular chaperones. Science 296, 2232-2235 (2002).

39. Reebye, V. et al. Role of the HSP90-associated cochaperone p23 in enhancing activity of the androgen receptor and significance for prostate cancer. Mol. Endocrinol. 26, 1694-1706 (2012).

40. Knoblauch, R. & Garabedian, M. J. Role for Hsp90-associated cochaperone p23 in estrogen receptor signal transduction. Mol. Cell. Biol. 19, 3748-3759 (1999).

41. Thul, P. J. et al. A subcellular map of the human proteome. Science 356, (2017).

42. Wu, H., Hyun, J., Martinez-Yamout, M. A., Park, S. J. & Dyson, H. J. Characterization of an Hsp90-Independent Interaction between Co-Chaperone p23 and Transcription Factor p53. Biochemistry 57, 935-944 (2018).

43. Zeng, P.-Y., Vakoc, C. R., Chen, Z.-C., Blobel, G. A. & Berger, S. L. In vivo dual cross-linking for identification of indirect DNA-associated proteins by chromatin immunoprecipitation. BioTechniques 41, 694,696,698 (2006).

44. Tan, C. S. H. et al. Thermal proximity coaggregation for system-wide profiling of protein complex dynamics in cells. Science 359, 1170-1177 (2018).

45. Parolia, A. et al. Distinct structural classes of activating FOXA1 alterations in advanced prostate cancer. Nature 571, 413-418 (2019).

46. Kregel, S. et al. Androgen receptor degraders overcome common resistance mechanisms developed during prostate cancer treatment. Neoplasia 22, 111-119 (2020).

47. Spratt, D. E. et al. Transcriptomic Heterogeneity of Androgen Receptor Activity Defines a de novo low AR-Active Subclass in Treatment Naïve Primary Prostate Cancer. Clin. Cancer Res. 25, 6721-6730 (2019).

48. Hallenbeck, K. K. et al. A Liquid Chromatography/Mass Spectrometry Method for Screening Disulfide Tethering Fragments. SLAS DISCOVERY: Advancing the Science of Drug Discovery 23, 183-192 (2018).

49. Ostrem, J. M., Peters, U., Sos, M. L., Wells, J. A. & Shokat, K. M. K-Ras(G12C) inhibitors allosterically control GTP affinity and effector interactions. Nature 503, 548-551 (2013).

50. McCormick, F. Sticking it to KRAS: Covalent Inhibitors Enter the Clinic. Cancer Cell 37, 3-4 (2020).

51. Sadowsky, J. D. et al. Turning a protein kinase on or off from a single allosteric site via disulfide trapping. PNAS (2011) doi:10.1073/pnas.1102376108.

52. Scheer, J. M., Romanowski, M. J. & Wells, J. A. A common allosteric site and mechanism in caspases. PNAS 103, 7595-7600 (2006).

53. Gao, J. & Wells, J. A. Identification of Specific Tethered Inhibitors for Caspase-5. Chemical Biology & Drug Design 79, 209-215 (2012).

54. Buck, E. & Wells, J. A. Disulfide trapping to localize small-molecule agonists and antagonists for a G protein-coupled receptor. PNAS 102, 2719-2724 (2005).

55. Buck, E., Bourne, H. & Wells, J. A. Site-specific disulfide capture of agonist and antagonist peptides on the C5a receptor. J. Biol. Chem. 280, 4009-4012 (2005).

56. Bulfer, S. L., Jean-Francois, F. L. & Arkin, M. R. Making FBDD Work in Academia. in Fragment-based Drug Discovery Lessons and Outlook 223-246 (John Wiley & Sons, Ltd, 2016). doi:10.1002/9783527683604.ch10.

1-15. (canceled)

16. An androgen receptor-fluorescent protein reporter system comprising:
a prostate cancer cell expressing a split fluorescent protein comprising a first component of a fluorescent protein and a second component of the fluorescent protein, wherein the prostate cancer cell comprises a modified genome comprising a first nucleic acid comprising a coding sequence encoding the first component of the fluorescent protein connected to a 5′ end of a protein coding exon of an androgen receptor (AR) gene such that the prostate cancer cell expresses a fusion protein comprising the first component of the fluorescent protein linked to the N-terminus of the androgen receptor; and
a vector comprising an expression cassette comprising a second nucleic acid comprising a coding sequence encoding the second component of the fluorescent protein, wherein a reconstituted fluorescent protein forms when the first component and the second component of the split fluorescent protein are expressed and interact to produce a detectable fluorescent signal indicative of the level of the androgen receptor in the prostate cancer cell.

17. The androgen receptor-fluorescent protein reporter system of claim 16, wherein the split fluorescent protein is a split yellow-green fluorescent protein.

18. The androgen receptor-fluorescent protein reporter system of claim 17, wherein the split yellow-green fluorescent protein is a split NeonGreen fluorescent protein.

19. The androgen receptor-fluorescent protein reporter system of claim 18, wherein the split NeonGreen fluorescent protein is a monomeric NeonGreen fluorescent protein split between the tenth β-strand and the eleventh β-strand.

20. The androgen receptor-fluorescent protein reporter system of claim 19, wherein the first component of the fluorescent protein consists of the eleventh β-strand of the monomeric NeonGreen fluorescent protein, and the second component of the fluorescent protein consists of the first β-strand through the tenth β-strand of the monomeric NeonGreen fluorescent protein.

21. The androgen receptor-fluorescent protein reporter system of claim 16, wherein the AR gene is an endogenous AR gene.

22. The androgen receptor-fluorescent protein reporter system of claim 16, wherein the vector is a lentivirus vector.

23. A prostate cancer cell comprising the androgen receptor-fluorescent protein reporter system of claim 16.

24. The prostate cancer cell of claim 23, wherein the prostate cancer cell is a LNCaP or a C42B prostate cancer cell.

25. A method of determining localization or measuring a level of an androgen receptor in a cell, the method comprising:
providing a prostate cancer cell expressing a split fluorescent protein comprising a first component of a fluorescent protein and a second component of the fluorescent protein, wherein the cell comprises a modified genome comprising a first nucleic acid comprising a coding sequence encoding the first component of the fluorescent protein connected to a 5′ end of a protein coding exon of an androgen receptor (AR) gene such that the prostate cancer cell expresses a fusion protein comprising the first component of the fluorescent protein linked to the N-terminus of the androgen receptor;
transfecting the prostate cancer cell with a vector comprising an expression cassette comprising a second nucleic acid comprising a coding sequence encoding the second component of the fluorescent protein, wherein a reconstituted fluorescent protein forms when the first component and the second component of the split fluorescent protein are expressed and interact to produce a detectable fluorescent signal; and
detecting the fluorescent signal, wherein the level or localization of the androgen receptor protein in the cell is determined from the fluorescent signal.

26. The method of claim 25, wherein the split fluorescent protein is a split yellow-green fluorescent protein.

27. The method of claim 26, wherein the split yellow-green fluorescent protein is a split NeonGreen fluorescent protein.

28. The method of claim 27, wherein the split NeonGreen fluorescent protein is a monomeric NeonGreen fluorescent protein split between the tenth β-strand and the eleventh β-strand.

29. The method of claim 28, wherein the first component of the fluorescent protein consists of the eleventh β-strand of the monomeric NeonGreen fluorescent protein, and the second component of the fluorescent protein consists of the first β-strand through the tenth β-strand of the monomeric NeonGreen fluorescent protein.

30. The method of claim 25, wherein the AR gene is an endogenous AR gene.

31. The method of claim 25, further comprising fixing the cell prior to said detecting the fluorescent signal.

32. The method of claim 25, wherein the fluorescent signal is detected by fluorescence microscopy.

33. The method of claim 25, further comprising contacting the cell with an agent that alters the localization or the level of the androgen receptor prior to said detecting the fluorescent signal.

34. The method of claim 33, wherein the agent is a small molecule, a peptide, a protein, an aptamer, an antibody, an antibody mimetic, an inhibitory nucleic acid, or a Clustered Regularly Interspaced Short Palindromic Repeats (CRISPR) system.

35. The method of claim 34, wherein the antibody is selected from the group consisting of a polyclonal antibody, a monoclonal antibody, a chimeric antibody, a humanized antibody, a F(ab) fragment, a F(ab′)2 fragment, a Fᵥ fragment, and a nanobody.

36. The method of claim 34, wherein the inhibitory nucleic acid is selected from the group consisting of a small interfering RNA (siRNA), a microRNA (miRNA), a Piwi-interacting RNA (piRNA), a small nuclear RNA (snRNA), an antisense oligonucleotide, and a peptide nucleic acid.

37. The method of claim 34, wherein the CRISPR system targets a gene or a RNA transcript, or makes epigenetic changes that alters the level of the androgen receptor.

38. The method of claim 37, wherein the CRISPR system comprises Cas9, Cas12a, Cas12d, Cas13a, Cas13b, Cas13d, or a dead Cas9 (dCas9).

39. The method of claim 34, wherein the agent is an inhibitor of prostaglandin E synthase 3 (PTGES3).

40. The method of claim 25, wherein the vector is a lentivirus vector.
